(19) United States
(12) Patent Application Publication
(10) Pub. No.: US 2026/0100259 A1
(43) Pub. Date: Apr. 9, 2026

(54) SYSTEMS AND METHODS FOR PREDICTING MENTAL HEALTH CONDITIONS BASED ON PASSIVE PROCESSING OF CONVERSATIONAL SPEECH AND LANGUAGE

(71) Applicant: Ellipsis Health, Inc., San Francisco, CA (US)

(72) Inventors: Elizabeth Shriberg, Berkeley, CA (US); Piotr Chlebek, Gdansk (PL); Tomasz Rutowski, Gdansk (PL); Amirhossein Haratinejadtorbati, Toronto (CA); Michael Aratow, Mountain View, CA (US); Mainul Mondal, San Francisco, CA (US); Melissa McCool, Encinitas, CA (US); Vaishnavi Narayanan, Austin, TX (US); Ishani Tagore, Austin, TX (US); Yang Lu, Waterloo, CA (US); Tulio Goulart, San Francisco, CA (US); Robert Rozanski, San Francisco, CA (US)

(73) Assignee: Ellipsis Health, Inc., San Francisco, CA (US)

(21) Appl. No.: 19/417,870

(22) Filed: Dec. 12, 2025

Related U.S. Application Data
(63) Continuation of application No. PCT/US2024/033710, filed on Jun. 13, 2024.
(60) Provisional application No. 63/507,973, filed on Jun. 13, 2023.
(60) Provisional application No. 63/571,398, filed on Mar. 28, 2024.

(51) Int. Cl. G16H 15/00; G10L 15/16; G16H 20/00; G16H 40/67

(52) U.S. Cl. CPC G16H 15/00; G10L 15/16; G16H 20/00; G16H 40/67

(57) ABSTRACT

Described herein are systems and methods for identifying the severity of a mental health condition or symptoms of same by listening to a human-to-human conversation by receiving conversation data, processing the conversation data to generate a language model output and/or an acoustic model output using one or more language models and/or acoustic models. Further described herein are systems and methods for automatically tracking and providing analytics on self-report questionnaires administered during the conversation.

### CROSS-REFERENCE

This application is a continuation of International Application No. PCT/US2024/033710, filed Jun. 13, 2024, which claims benefit and priority from U.S. Provisional Patent Application No. 63/507973, entitled “SYSTEMS AND METHODS FOR PREDICTING CONDITIONS BASED ON CONVERSATIONS”, filed on 13 Jun. 2023, and U.S. Provisional Patent Application No. 63/571398, entitled “SYSTEMS AND METHODS FOR PREDICTING MENTAL HEALTH CONDITIONS BASED ON PASSIVE PROCESSING OF CONVERSATIONAL SPEECH AND LANGUAGE”, filed on 28 Mar. 2024, the contents of which applications are incorporated by reference.

### FIELD

The present disclosure generally relates to the field of health assessments, and more specifically, embodiments relate to devices, systems, and methods using artificial intelligence to predict a patient's severity of symptoms of mental health conditions based on conversations with healthcare providers.

### INTRODUCTION

Behavioral health is a serious concern. The most widely used tools for screening for behavioral health conditions may rely on accurate self-reporting by the screened patient. For example, the current “gold standard” for questionnaire-based screening for depression is the Patient Health Questionnaire-9 (PHQ-9), a written depression health survey with nine multiple-choice questions. Other similar health surveys include the Patient Health Questionnaire-2 (PHQ-2) and the Generalized Anxiety Disorder 7 (GAD-7).

These and other screening or monitoring surveys may not be engaging due to their repetitive nature and lack of personalization. Because they are self reported, they may also lack an element of objectivity. It may be difficult to assess a patient's behavioural health if no assessment questionnaire is provided or if the survey questions are incomplete. Patients may begin to answer questions in a rote manner after receiving the same questions over multiple sessions making assessment of patient progress difficult. Patients may also answer the survey in a biased fashion to influence their therapist or hide a condition for fear of job loss or discrimination.

Finally, it takes effort on the part of the agent (e.g., a healthcare team member, case manager, etc.) and the patient to complete these surveys (e.g., some patients may need assistance) and this disrupts both the agent and patient workflows. Frequently these surveys are verbally administered, which can take up a substantial portion of a clinical encounter. In addition, when these surveys are verbally administered, they frequently are done incorrectly (e.g., not asking all the questions, not asking the questions verbatim, not providing the correct choices for answers), essentially invalidating their results. This can lead to increased healthcare costs incurred by the patients, providers, and payers. In some cases, it may also lead to poorer patient outcomes (e.g., if it prevents or delays a patient from being properly seen due to incorrect stratification from an erroneous score).

Improvement in the field of patient case/care management is needed.

### SUMMARY

Screening or monitoring surveys may not be engaging due to their repetitive nature and lack of personalization, and not always objective as they are self reported. This may further be exacerbated if these questions are delivered in written survey form or by an automatic question delivery system. It may be more engaging for patients if questionnaire questions were delivered by a human (e.g., an agent) responsive to the patient's context. It may further be beneficial if answers to the questions could be pulled from existing conversations with the patient.

Agents may be too busy speaking with the patient to simultaneously pull relevant discussion points out from the conversation. Furthermore, the agent may not be able to pull out relevant answers in a timely manner or even at all. A robust, and automatic system or method to passively listen to agent-patient conversations to pull out relevant answers or other relevant information is needed.

It may be difficult to assess a patient's behavioural health if no assessment questionnaire is provided or if the survey questions are incomplete. Agents may be able to pull out relevant responses from patients using a conversational approach which may make progress assessment more straightforward. Systems and methods described herein may further be helpful to pull relevant topics and relevant sections of conversation to predict a patient's condition. Systems and methods described herein can harness already existing conversations to make an assessment, obviating the need for doing the verbatim assessment.

Finally, it takes effort on the part of the clinical team member and the patient to complete surveys (e.g., some patients need assistance for their completion) and this disrupts both the agent and patient workflows. Providing a system or method to do the work of session analysis, transcription, and annotation can help the agent provide prompt (e.g., real-time or just-in-time), accurate, and precise referral and treatment. This may not only lead to better patient outcomes, but it may further free up the agent's capacity to assess more patients in a shorter period of time or spend the saved time addressing other patient concerns or needs.

According to an aspect, there is provided a system to analyze a conversation (speech and/or text) to predict severity of symptoms. The system including at least one input device for receiving conversation data from at least one user, at least one output device for outputting an electronic report, and at least one computing device in communication with the at least one input device and the at least one output device. The at least one computing device is configured to receive the conversation data from the at least one input device, process the conversation data to generate a language model output and/or an acoustic model output, optionally fuse the language model output and the acoustic model output by applying weights to the language model output and the acoustic model output and generating a composite output from the weighted outputs, generate an electronic report, and transmit the electronic report to the output device.

According to an aspect, there is provided a system for identifying roles of speakers in a conversation. The system including at least one input device for receiving conversation data from at least one user, at least one output device for outputting an electronic report, and at least one computing device in communication with the at least one input device and the at least one output device. The at least one computing device is configured to receive the conversation data from the at least one input device, determine at least one role of at least one speaker, process the conversation data to generate a language model output and/or an acoustic model output, applying weights to the language model output and/or the acoustic model output, wherein the language model output and the acoustic model output each comprise a plurality of outputs corresponding to a plurality of time segments of the conversation data, wherein the weights are optionally temporally-based, and wherein the weights are based in part on the at least one role of the at least one speaker during each time segment, and generate an electronic report, and transmit the electronic report to the output device.

In some embodiments, the at least one computing device is further configured to fuse the weighted language model output and the acoustic model output generating a composite output. The composite output may represent the fused output from the fusion of the language model output and the acoustic model output.

In some embodiments, the electronic report may identify a severity of at least one symptom of a condition based on the composite output.

In some embodiments, the condition may comprise a mental health condition.

In some embodiments, the electronic report comprises an annotation of the language model output indicating salience of at least one of the one or more time segments.

In some embodiments, the at least one speaker comprises an agent and applying the weights to the language model output and the acoustic model output comprises applying a zero weight to acoustic model output corresponding to the agent.

In some embodiments, the weights are based in part on a topic during each time segment.

In some embodiments, the language model output comprises an identification of at least one query based on conversation data from an agent and at least one response to the at least one query based on the conversation data from a patient, wherein the at least one query is mapped onto a predefined query and the at least one response to the at least one query is mapped onto a predefined response to the predefined query.

In some embodiments, processing the conversation data to generate the language model output and the acoustic model output comprises using a language model neural network and an acoustic neural network trained on labelled conversation data collected from one or more other subjects, wherein the labelled conversation data comprises is labelled as (i) having, to some level, a condition and (ii) not having the condition.

In some embodiments the at least one role of the at least one speaker includes at least one of a patient, an agent, an interactive voice response, and bot speaker.

In some embodiments, the weights applied to the language model output and the acoustic model output are based in part on determining that a number of the at least one speaker matches an expected number of speakers.

In some embodiments, the conversation data is processed based in part on at least one of a patient profile and an agent profile, wherein the profile comprises at least one of historical, biographical, demographic, and longitudinal data.

In some embodiments, the conversation data includes at least one of speech data and text-based data.

In some embodiments, the at least one computing device is configured to run a model based on human-interpretable features.

According to an aspect there is provided a system for identifying topics in a conversation. The system including at least one input device for receiving conversation data from at least one user, at least one output device for outputting an electronic report, at least one computing device in communication with the at least one input device and the at least one output device. The at least one computing device is configured to receive the conversation data from the at least one input device, process the conversation data to generate a language model output, wherein the language model output comprises one or more topics corresponding to one or more time ranges, apply weights to an output to generate a weighted output, wherein the output comprises a plurality of outputs corresponding to a plurality of time segments of the conversation data, wherein the weights are optionally temporally-based, and wherein the weights are based in part on the one or more topics during each time segment, generate an electronic report, and transmit the electronic report to the output device.

In some embodiments, the at least one computing device is configured to process the conversation data to generate an acoustic model output, and fuse the language model output and the acoustic model output by applying weights to the language model output and the acoustic model output and generating a composite output from the weighted outputs, wherein the acoustic model output comprises a plurality of outputs corresponding to a plurality of time segments of the conversation data, wherein the weights are optionally temporally-based, and wherein the weights are based in part on the topic during each time segment.

In some embodiments, time ranges of the conversation data corresponding to a predefined topic are processed to generate the language model output using more computationally robust models than those used for time ranges of the conversation data not corresponding to the predefined topic.

In some embodiments, the electronic report comprises a transcript of the language model output annotated based in part on the weights based in part on the topic during each time segment.

In some embodiments, the electronic report identifies a severity of at least one symptom of a condition based on the language model output.

In some embodiments, the condition comprises a mental health condition.

In some embodiments, the electronic report comprises an annotation of the language model output indicating salience of at least one of the one or more time segments.

In some embodiments, the computing device is further configured to determine at least one role of at least one speaker and wherein the weights are based in part on the at least one role of the at least one speaker during each time segment.

In some embodiments, the language model output comprises an identification of at least one query based on conversation data from an agent and at least one response to the at least one query based on the conversation data from a patient, wherein the at least one query is mapped onto a predefined query and the at least one response to the at least one query is mapped onto a predefined response to the predefined query.

In some embodiments, processing the conversation data to generate the language model output comprises using a language model neural network trained on labelled conversation data collected from one or more other subjects, wherein the labelled conversation data comprises is labelled as (i) having, to some level, a condition and (ii) not having the condition.

In some embodiments, the at least one query is of a set of queries and the computing device is configured to predict an overall score based on the set of queries based on responses to each of the queries of the set of queries.

In some embodiments, the conversation data is processed based in part on at least one of a patient profile and an agent profile, wherein the profile comprises at least one of historical, biographical, demographic, and longitudinal data.

In some embodiments, the conversation data comprises at least one of speech data and text-based data.

In some embodiments, the at least one computing device is configured to run a model based on human-interpretable features.

According to an aspect, there is provided a system for scoring surveys based on a conversation. The system includes at least one input device for receiving conversation data from at least one user, at least one output device for outputting an electronic report, at least one computing device in communication with the at least one input device and the at least one output device. The at least one computing device is configured to receive the conversation data from the at least one input device, process the conversation data to generate a language model output, wherein the language model output comprises an identification of at least one query based on conversation data from an agent and at least one response to the at least one query based on the conversation data from a patient, wherein the at least one query is mapped onto a predefined query and the at least one response to the at least one query is mapped onto a predefined response to the predefined query, and generate an electronic report, and transmit the electronic report to the output device.

In some embodiments, the at least one computing device is further configured to process the conversation data to generate an acoustic model output and fuse the language model output and the acoustic model output by applying weights to the language model output and the acoustic model output and generating a composite output from the weighted outputs, wherein the language model output and the acoustic model output each comprise a plurality of outputs corresponding to a plurality of time segments of the conversation data, wherein the weights are optionally temporally-based.

In some embodiments, the weights are based in part on the at least one query or a topic during each time segment.

In some embodiments, the system is configured to output at least one outstanding query on the output device. The outstanding query may be a query to which the user has not provided a response.

In some embodiments, the system is configured to output a flag indicating that the at least one response to the at least one query is not mapped onto a predefined response to the predefined query with confidence exceeding a threshold.

In some embodiments, the system is configured to output a prompt to repeat the predefined query when it is determined that the at least one response to the at least one query is not mapped onto a predefined response to the predefined query with confidence exceeding a threshold.

In some embodiments, the electronic report identifies a severity of at least one symptom of a condition based on the language model output.

In some embodiments, the condition comprises a mental health condition.

In some embodiments, the electronic report comprises an annotation of the language model output indicating salience of at least one of the one or more time segments.

In some embodiments, the computing device is further configured to determine at least one role of at least one speaker and wherein the weights are based in part on the at least one role of the at least one speaker during each time segment.

In some embodiments, processing the conversation data to generate the language model output comprises using a language model neural network trained on labelled conversation data collected from one or more other subjects, wherein the labelled conversation data comprises is labelled as (i) having, to some level, a condition and (ii) not having the condition.

In some embodiments, the conversation data is processed based in part on at least one of a patient profile and an agent profile, wherein the profile comprises at least one of historical, biographical, demographic, and longitudinal data.

In some embodiments, the conversation data comprises at least one of speech data and text-based data.

In some embodiments, the at least one computing device is configured to run a model based on human-interpretable features.

According to an aspect there is provided a method for identifying roles of speakers in a conversation. The method including receiving conversation data from at least one input device, determining at least one role of at least one speaker, processing the conversation data to generate a language model output and/or an acoustic model output, applying weights to the language model output and/or the acoustic model output, wherein the language model output and/or the acoustic model output each comprise a plurality of outputs corresponding to a plurality of time segments of the conversation data, wherein the weights are optionally temporally-based, and wherein the weights are based in part on the at least one role of the at least one speaker during each time segment, generating an electronic report, and transmitting the electronic report to an output device.

In some embodiments, the further includes fusing the weighted language model output and the acoustic model output generating a composite output. The composite output may represent the fused output from the fusion of the language model output and the acoustic model output.

In some embodiments, the electronic report identifies a severity of at least one symptom of a condition based on the composite output.

In some embodiments, the condition comprises a mental health condition.

In some embodiments, the electronic report comprises an annotation of the language model output indicating salience of at least one of the one or more time segments.

In some embodiments, the at least one speaker comprises an agent and applying the weights to the language model output and the acoustic model output comprises applying a zero weight to acoustic model output corresponding to the agent.

In some embodiments, the weights are based in part on a topic during each time segment.

In some embodiments, the language model output comprises an identification of at least one query based on conversation data from an agent and at least one response to the at least one query based on the conversation data from a patient, wherein the at least one query is mapped onto a predefined query and the at least one response to the at least one query is mapped onto a predefined response to the predefined query.

In some embodiments, processing the conversation data to generate the language model output and the acoustic model output comprises using a language model neural network and an acoustic neural network trained on labelled conversation data collected from one or more other subjects, wherein the labelled conversation data comprises is labelled as (i) having, to some level, a condition and (ii) not having the condition.

In some embodiments, the at least one role of the at least one speaker comprises at least one of a patient, an agent, an interactive voice response speaker, and a bot speaker.

In some embodiments, the weights applied to the language model output and the acoustic model output are based in part on determining that a number of the at least one speaker matches an expected number of speakers.

In some embodiments, the conversation data is processed based in part on at least one of a patient profile and an agent profile, wherein the profile comprises at least one of historical, biographical, demographic, and/or longitudinal data.

In some embodiments, the conversation data comprises at least one of speech data and text-based data.

In some embodiments, the at least one computing device is configured to run a model based on human-interpretable features.

According to an aspect, there is provided a method for identifying topics in a conversation. The method includes receiving conversation data from at least one input device, processing the conversation data to generate a language model output, wherein the language model output comprises one or more topics corresponding to one or more time ranges, applying weights to an output to generate a weighted output, wherein the output comprises a plurality of outputs corresponding to a plurality of time segments of the conversation data, wherein the weights are optionally temporally-based, and wherein the weights are based in part on the one or more topics during each time segment, generating an electronic report, and transmitting the electronic report to an output device.

In some embodiments, the method further comprises processing the conversation data to generate an acoustic model output and fusing the language model output and the acoustic model output by applying weights to the language model output and the acoustic model output and generating a composite output from the weighted outputs, wherein the acoustic model output comprises a plurality of outputs corresponding to a plurality of time segments of the conversation data, wherein the weights are optionally temporally-based, and wherein the weights are based in part on the topic during each time segment.

In some embodiments, time ranges of the conversation data corresponding to a predefined topic are processed to generate the language model output using more computationally robust models than those used for time ranges of the conversation data not corresponding to the predefined topic.

In some embodiments, the electronic report comprises a transcript of the language model output annotated based in part on the weights based in part on the topic during each time segment.

In some embodiments, the electronic report identifies a severity of at least one symptom of a condition based on the language model output.

In some embodiments, the condition comprises a mental health condition.

In some embodiments, the electronic report comprises an annotation of the language model output indicating salience of at least one of the one or more time segments.

In some embodiments, further comprising determining at least one role of at least one speaker and wherein the weights are based in part on the at least one role of the at least one speaker during each time segment.

In some embodiments, the language model output comprises an identification of at least one query based on conversation data from an agent and at least one response to the at least one query based on the conversation data from a patient, wherein the at least one query is mapped onto a predefined query and the at least one response to the at least one query is mapped onto a predefined response to the predefined query.

In some embodiments, processing the conversation data to generate the language model output comprises using a language model neural network trained on labelled conversation data collected from one or more other subjects, wherein the labelled conversation data comprises is labelled as (i) having, to some level, a condition and (ii) not having the condition.

In some embodiments, the at least one query is of a set of queries and the computing device is configured to predict an overall score based on the set of queries based on responses to each of the queries of the set of queries.

In some embodiments, the conversation data is processed based in part on at least one of a patient profile and an agent profile, wherein the profile comprises at least one of historical, biographical, demographic, and longitudinal data.

In some embodiments, the conversation data comprises at least one of speech data text-based data.

In some embodiments, the method is configured to run a model based on human-interpretable features.

According to an aspect, there is provided a method for scoring surveys based on a conversation. The method includes receiving conversation data from at least one input device, processing the conversation data to generate a language model output, wherein the language model output comprises an identification of at least one query based on conversation data from an agent and at least one response to the at least one query based on the conversation data from a patient, wherein the at least one query is mapped onto a predefined query and the at least one response to the at least one query is mapped onto a predefined response to the predefined query, generating an electronic report, and transmitting the electronic report to an output device.

In some embodiments, the method further includes processing the conversation data to generate an acoustic model output, and fusing the language model output and the acoustic model output by applying weights to the language model output and the acoustic model output and generating a composite output from the weighted outputs, wherein the language model output and the acoustic model output each comprise a plurality of outputs corresponding to a plurality of time segments of the conversation data, wherein the weights are optionally temporally-based.

In some embodiments, the weights are based in part on the at least one query or a topic during each time segment.

In some embodiments, the method further includes outputting at least one outstanding query on the output device. The outstanding query may be a query to which the user has not provided a response.

In some embodiments, the method further includes outputting a flag indicating that the at least one response to the at least one query is not mapped onto a predefined response to the predefined query with confidence exceeding a threshold.

In some embodiments, the method further includes outputting a prompt to repeat the predefined query when it is determined that the at least one response to the at least one query is not mapped onto a predefined response to the predefined query with confidence exceeding a threshold.

In some embodiments, the electronic report identifies a severity of at least one symptom of a condition based on the language model output.

In some embodiments, the condition comprises a mental health condition.

In some embodiments, the electronic report comprises an annotation of the language model output indicating salience of at least one of the one or more time segments.

In some embodiments, the method further comprises determining at least one role of at least one speaker and wherein the weights are based in part on the at least one role of the at least one speaker during each time segment.

In some embodiments, processing the conversation data to generate the language model output comprises using a language model neural network trained on labelled conversation data collected from one or more other subjects, wherein the labelled conversation data comprises is labelled as (i) having, to some level, a condition and (ii) not having the condition.

In some embodiments, the conversation data is processed based in part on at least one of a patient profile and an agent profile, wherein the profile comprises at least one of historical, biographical, demographic, and longitudinal data.

In some embodiments, the conversation data comprises at least one of speech data and text-based data.

In some embodiments, the method configured to run a model based on human-interpretable features.

According to an aspect, there is provided a non-transient computer readable medium containing program instructions for causing a computer to perform any of the above methods.

According to an aspect, there is provided a system to train a baseline for one or more of a language model, an acoustic model, and a fusion model (the model) to directly or indirectly detect a behavioural or mental health condition using machine learning. The training includes predicting the behavioural or mental health condition in training data using the model, updating the model based on accuracy of the prediction.

In some embodiments, labels in training data are assessed for trustworthiness before or during training and each training datum is reweighed according to the trustworthiness of its label.

In some embodiments, training data is augmented using at least one of paraphrasing or synthetic speech to generate additional training data for use in the training data.

In some embodiments, the training includes predicting a speaker ID and penalizing the model for accurately identifying the speaker ID. In some embodiments, the model will be penalized for learning information about the speaker ID rather than about the speaker's state.

According to an aspect, there is provided a system for predicting a severity of at least one symptom of a behavioral or mental health condition of a subject. The system including at least one input device for receiving conversation data from the subject and at least one computing device in communication with the at least one input device. The at least one computing device is configured to receive in-context learning comprising an explanation related to one or more questions of a questionnaire, receive the conversation data from the at least one input device, and predict the severity of the at least one symptom of the behavioral or mental health condition of the subject based on the in-context learning and the conversation data.

In some embodiments, the computing device accesses a large language model to predict the severity of the at least one symptom of the behavioral or mental health condition of the subject based on the in-context learning and the conversation data.

In some embodiments, the prediction of the severity of the at least one symptom of the behavioral or mental health condition comprises a prediction of a result of the questionnaire.

In some embodiments, the prediction of the severity of the at least one symptom of the behavioural or mental health condition comprises a prediction of a result of at least one of the one or more questions of the questionnaire.

According to an aspect, there is provided system for pre-processing transcript data for use in predicting a severity of at least one symptom of a behavioral or mental health condition of a subject. The system comprising at least one input device for receiving conversation data from the subject and at least one computing device in communication with the at least one input device. The at least one computing device is configured to receive in-context learning comprising a description of the behavioral or mental health condition, receive the conversation data from the at least one input device, pre-process the conversation data by performing at least of weighing at least one segment of the conversation data based on a relation between the at least one segment of the conversation data and the behavioral or mental health condition, summarizing the at least one segment of the conversation data, providing analytics on the at least one segment of the conversation data, summarizing at least one aspect of the behavioral or mental health condition, and providing analytics on the at least one aspect of the behavioral or mental health condition, and transmit the pre-processed conversation data to one or more models to predict the behavioral or mental health condition of the subject.

In some embodiments, the computing device accesses a large language model to pre-process the conversation data.

Many further features and combinations thereof concerning embodiments described herein will appear to those skilled in the art following a reading of the instant disclosure.

### DESCRIPTION OF THE FIGURES

In the figures,

FIG. 1 illustrates an example system for predicting a patient condition based on passive listening, according to some embodiments.

FIG. 2 illustrates a method of pre-processing the speech data, according to some embodiments.

FIG. 3 illustrates a method of detecting roles of speakers, according to some embodiments.

FIG. 4 illustrates a method of detecting topics within the conversation, according to some embodiments.

FIG. 5 illustrates a method to score a survey based on passively listening to a conversation, according to some embodiments.

FIG. 6 illustrates an example UI for case managers, according to some embodiments.

FIG. 7 illustrates an example UI for case managers while calling a patient, according to some embodiments.

FIG. 8 illustrates an example UI for case managers while the system is delivering a behavioural health score, according to some embodiments.

FIG. 9 illustrates an example call summary of a care meeting, according to some embodiments.

FIG. 10 illustrates a schematic diagram of computing device, according to some embodiments.

### DETAILED DESCRIPTION

Provided herein are systems and methods that can passively listen to a conversation including a patient and analyze the conversation to estimate the patient's likelihood of mental health conditions using acoustic and language models. The conversation could be between the patient and, for example, an agent such as a healthcare team member, a case manager, etc. The conversation could be between a patient and a bot (e.g., a chatbot or an AI agent). The systems and methods described herein may work on different forms of conversation data such as spoken (e.g., verbal conversations) or written (e.g., taking place by text messaging) conversations. The systems and methods described herein may work on utterances or textual information provided by one user (e.g., spontaneous utterances of a patient or analysis of journal entries, which may still be referred to as “conversation data”). The systems and methods described herein may work with non-contemporaneous conversation data (e.g., conversations that take place over time with gaps between each turn in the conversation, e.g., conversations by text). The systems and methods may include Role Detection, diarization, Topic Detection to aid in assessment and computational analysis, and Survey Scoring to aid in agent assessment and diarization. The systems and methods described herein may make use of natural language processing (NLP) and/or acoustic models to analyze the conversation. The systems and methods described herein may be useful for non-experts in, for example, mental health to make assessments and provide clinical decision support.

Aspects of the systems and methods described herein produce severity of symptoms of mental health conditions from existing human-to-human conversations. The systems and methods described herein can address the over- or under-detection of mental health conditions, and the inadequacy of reliance on self-report instruments such as the PHQ-9 for this purpose. There may be a high risk of depression and anxiety in populations with chronic conditions and these patients may participate in regular calls with agents, but these agents may not be trained in mental health. The administration of self-report screening tools may not be adequate in the context, for many reasons such as lack of consistency in using and administering, lack of engagement, extra time, repetition etc. Assessing these casual conversations may be beneficial.

In some embodiments, the systems and method described herein may further be advantageous as it may be better able to identify and account for over- or under-reporting. Some patient populations may over- or under-report on self-reported mental health surveys. For example, individuals who lived through WWII may be under-reporters while individuals of 25 years or less may be over-reporters. The systems and methods described herein may be capable of providing more accurate scoring for these individuals as the system can score patients with the benefit of the training data from whole population of individuals. Furthermore, the systems and methods described herein may be used to predict mental health status for populations (or subpopulations) of patients based on, for example, their demographics.

Furthermore, the systems and methods described herein may be configured to use data (e.g., demographic data, metadata, etc.) to evaluate the likelihood of a patient to over- or under-report and adjust the weightings of the model accordingly. For example, the model may be configured to weigh signs of depression more highly in under-reporters to account for their tendency to underreport.

It may be beneficial to assess a patient's state based not just on, for example, their responses to survey questions, but also based on their free-form conversation and based on the acoustic features of that conversation. Such an analysis may involve an assessment of relevant topics of conversation based on recognition of the language and assessment of the acoustic characteristics of the conversation. Furthermore, reserving sophisticated analysis of the conversation for segments of the conversation relating to relevant topics may make more economically efficient use of computational resources. Such features may be of particular use when delivering real-time feedback or when using third-party paid models (e.g., to use more sophisticated and more expensive models only on the most relevant portion of the conversation).

In some embodiments, the systems and methods described herein can be used to passively listen to the conversations between a patient and a healthcare professional. Unlike with an agent, the healthcare professional may have a more improvisational speech. The questions asked may flag particular conditions, but may not, in total, map onto a conventional questionnaire. Diarization and text summary may aid the healthcare professional in meeting their record keeping requirements without necessitating additional work from the healthcare professional.

Additionally, the actual administration of PROs (Patient Reported Outcomes) may be done with varying competency by agents, who may not ask questions verbatim, fail to ask all the questions, or input the wrong patient answer using one or the different choices with which the PRO was validated.

The systems and methods described herein harness regular conversations between an agent and a patient to provide an expert “ear” that has been trained to listen for evidence of mental health symptoms in speech and language. The invention may not require any additional effort or time from either party.

Some technical advantages of the systems and methods described herein include improved mental health symptom prediction over conventional systems and methods, prediction in less overall time, improved rapport between patient and agent, more tailored content for the patient, more accurate scoring of verbally administered surveys, improved compliance with sets of questions to assess patients, and improved triage and referral of patients (leading to improved outcomes).

In some embodiments, the systems and methods described herein may analyze conversations in real time, and provide clues/hints on how to improve it to an agent while the conversation is still ongoing (e.g., what to ask next, what to discuss deeper, which topics we should cover, etc.). Such implementations may further guide an agent to provide advice to a patient (e.g., how to deal with difficult times).

In some embodiments, the systems and methods described herein can be implemented without disrupting or lengthening the conversation between patient and agent. There may further be little to no additional training required for the agent. The outputs of the system may be usable “as is”, for example, for severity of symptoms of mental health condition predictions. In some embodiments, the systems and methods may be able to determine which speaker is the agent and which is the patient based on no training with that agent or that patient and analyze the conversation accordingly. In some embodiments the system may run just-in-time analysis, producing a severity of symptoms of mental health condition score at or before the end of a naturally occurring case/care management conversation. In some embodiments the symptom score, a survey score from a verbal survey administration, or a summary via automatic summarization, may be outputs of the systems and methods. Automatic summarization may be of interest as it decreases the amount of downtime required for each agent for documentation requirements. The mental health score may be connected to the organization's existing clinical pathways to provide the correct referral or recommendations commensurate with the mental health scores.

Surveys may be made more engaging when delivered by an agent rather than by a written survey or through an automatic delivery system. To make the conversation more free flowing, it may be beneficial to empower the agent to proceed through the questions of a survey as they become relevant to the free flowing conversation. It may also be beneficial to empower the agent to rephrase the questions based on their natural presentation and/or based on patient context.

Furthermore, it would be beneficial if a system was configured to pull out relevant answers to survey questions in spite of any difference in order of question delivery or difference in content of the question. Such a system would free the agent's time to see more patients during a working day. Furthermore, it may empower the agent to provide low-latency (e.g., real time or just-in-time), accurate, and precise referrals and treatment.

Conditions can be comorbid. Systems and methods described herein may take a prior diagnosis of one condition or a prediction by the system as an input to determine the presence of another condition. There may be different subtypes and different treatments on different subtypes (e.g., patients that may respond well to therapy v SSRI v TMS, anhedonia in depression, etc.). In treating the whole person, the more information, the better the system will be to recommend the appropriate treatment for a different subtype.

In some embodiments, the system may be configured to score the patients with regards to the severity of their conditions. For example, this score could be related to the confidence metric. This score could be used to determine triage among patients, for example, when healthcare resources throughout the system are low, the system may ensure that every person who exhibits depression is referred to therapy, however those with more serious cases are seen ahead of those with milder or more stable cases. Further, the system may be configured to recommend more aggressive and urgent treatment of those the system predicts confidently are suffering from a serious and urgent condition.

In some embodiments, the patient's reaction to a drug may inform the progression path of the condition (e.g., the system may monitor many patients on the drug to ascertain the likely progression path of a patient's condition while on the drug and apply that path to make predictions for further patients).

In some embodiments, the system may be configured to store longitudinal information about patients in, for example, a patient profile. In some embodiments, the system can measure the patient's risk under different drugs or treatments, dosages, and timelines. The system may be configured to predict whether a drug is working for a specific patient over time (rather than cycling the patient). By measuring session-to-session changes in conversation data, the system may be able to ascertain earlier and more accurately than a survey that a drug is effective. The system may also be able to use session-to-session changes to help with drug dosing and other adaptations. The system may also be configured to extract side-effect data from the sessions.

Surveys which patients complete may become rote and the patient's answers may be based more on their habitual answers rather than on actual self-reflection. As such, surveys may require a greater effective result to present as effective through survey measurements than measurements taken herein. There may be many reasons why answers in self-reports are unreliable. In some experiments, very few patients are capable of reliably repeating their answers. Surveys can also be completed based upon perceived gain by the patient or to elicit a certain response by those interpreting the survey results (e.g., pleasing the therapist because the patient is supposedly doing better).

Though described primarily in the context of a drug, the above usage may be equally applicable to any treatment scheme or other compliance regime. For example, in some embodiments, the treatment is an exercise routine, a nutrition plan, cognitive behavioural therapy, or mindfulness exercises. In some embodiments, the treatment plan is a combination of two or more elements (e.g., a drug and a nutrition plan). Any type of treatment or therapy may be compatible with this system.

### General System

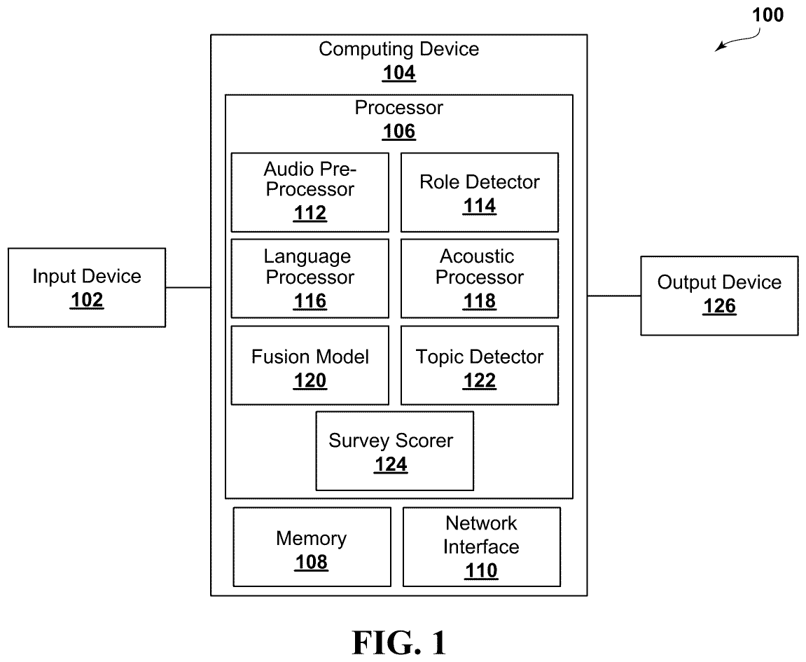

FIG. 1 illustrates an example system 100 for predicting a patient condition based on passive listening, according to some embodiments. The system 100 includes an input device 102, a computing device 104, and an output device 126.

### Input 102

The input device 102 may be configured to receive and relay data including, for example, audio data of a conversation between a patient and an agent (e.g., conversation data). The input device 102 can capture audio data from a conversation. For example, the input device may be a voice recorder present in a room, a passive listening device configured to listen to a telephone (or other remote) call, or another capture device methodology. In some embodiments, the input device 102 may include one or more of a microphone or other audio capture device passively listening to a one-on-one in-person session (e.g., between a physician and a patient) or an in-person session in a room potentially with other people (e.g., ambient microphones in a hospital setting or in a home health visit), landlines, cellphones, conference calling services, video conference calling services, call centers, etc. In some embodiments, the input device 102 may be any device configured to listen to a patient having a conversation, for example, with an agent.

In some embodiments, the input device 102 may use a single channel to capture a two-way conversation. In some embodiments, the input device 102 may use multiple channels to capture a conversation. In some embodiments, the input device 102 may be configured with a keyboard, mouse, camera, touch screen and/or a microphone to receive inputs from the agent or the patient. It may be beneficial for systems to be speaker-independent (i.e., may not have a baseline of the patient's speech).

In some embodiments, the input device 102 may include a channel from a care manager in a call center and a channel from a patient that the care manager is calling. In some embodiments, the care manager and patient may be on the same channel. In some embodiments, the care manager may be located in a call center and their input device may include further functionality to review patient data and or past conversations (including call notes).

In some embodiments, the systems and methods described herein could be implemented with (or supported by) a large language model-based chat bot (e.g., ChatGPT) or other generative speech/text scheme instead of a live agent. In such embodiments, the system may be configured to prompt the patient for certain responses, but also be trained to prompt responses that evoke longer responses from the user.

In some embodiments, these large language model-based chat bots may be used in the collection of training data. For example, the models may be implemented to simulate real agents and/or real patients to augment training data for one or more aspects of the system.

In some embodiments, the large language model-based chat bot may be supported by a virtual avatar. The virtual avatar may emote or otherwise exhibit backchannel communication in addition to prompting the patient for responses. This may be advantageous to automate the role of the agent while still providing a virtual human connection for the patient. The avatar may further be configured for a digital, augmented, and/or virtual reality system.

In some embodiments, the input device 102 may additionally receive additional input from the conversation. For example, in some embodiments, the input device 102 may be configured to capture visual data from one or more of the conversation participants (e.g., the patient). In such embodiments, the visual data may be used to detect visual indications of mental conditions to further enhance the confidence of the system.

In some embodiments, the input device 102 may include video input. For example, the session may be conducted over a video conference calling service and instead of or in addition to audio data, the system may be supplied with video data. In such circumstances, the model may be trained to assess the video data for cues that might be probative into the patient's mental condition or probative to regions of the session which may be of higher predictive value (e.g., topic detection, described in greater detail below). For example, the system may assess the patient's eye position, posture, gaze, or headmode and make assessments about the patient based on this information. In some embodiments, it may assess importance of regions of the session based on the video data (e.g., the system may weigh a portion of the session more highly when the patient spontaneously exhibited withdrawn body language or increased their self-soothing behaviours as it may indicate a portion of the session to which the patient is experiencing a significant emotional reaction). In some embodiments, the video data, may be used, for example, the aide the system in conversation data processing. For example, the system may track whose mouth is moving during turns of the conversation to better diarize the session. For example, if the patient is on a video call with a caretaker, then the system may be able to better distinguish from patient speech and caretaker speech by detecting whose mouth is moving. As a further example, conversation data may vary slightly if the patient is lying down and using video, the system may be able to account for this change.

In some embodiments, the system 100 may be configured to track conversations which occur via an SMS service or other text-based exchange (e.g., using such devices as the input device 102). In such embodiments, the model may be specifically trained to assess the textual habits of patients. The communication styles exhibited by patients via text may vary substantially from the communication styles exhibited in a voice chat. As such the models may need to be specifically trained to assess conditions in a textual environment. Furthermore, text message conversations may be less synchronous (e.g., the patient is doing something else) or entirely asynchronous (e.g., the agent and patient respond at random intervals throughout the day) which may dramatically impact the manner in which the data is analyzed. For example, it may more appropriate to treat each asynchronous exchange in a more isolated manner as the patient's context may vary in the time between exchanges due to many factors (which may be related to their mental condition, e.g., BPD, or which may not be related to their mental condition, e.g., burnt self on food).

Conversations which occur via text-based exchanges may be more important for individuals from certain demographic groups. For example, older individuals may be less prone to texting and treat it more like formal correspondence while younger individuals may be more comfortable sharing cues into their mental condition via text exchanges rather than in person. Furthermore, different subsets of the population may have different etiquette rules surrounding text-based communication (e.g., interpreting the use of a period as a sign of hostility among younger individuals). As a further complication of text-based communication, the use of emojis within text may have vastly different meanings depending on how it is used in the message and the demographics of the person using the emoji (e.g., the “blowing a kiss” emoji may be used in an endearing manner by older individuals, but in a sarcastic manner by younger individuals or depending on the context).

The use of text-based communication may be used instead of speech data. The use of text-based communication may be used in addition to the speech data. For example, the patient may text the agent infrequently between formal audio sessions either at, for example, the insistence of the agent or the leisure of the patient. The topics discussed during text conversations may be used to inform the analysis of the speech data. For example, the patient or the agent may refer back to topics discussed via text and the system may be configured to analyze the text message conversations to retrieve the topic discussed.

In some embodiments, the system may be configured to assess the mental condition of one or more patients in a group text.

In some embodiments, the input device 102 may pull relevant information from other resources. For example, the input device 102 may be configured to retrieve biographical or other patient data (e.g., contained in an Electronic Health Record (EHR)) from an external source to provide to the computing device 104 to enhance the predictive capacity of the system 100. Beyond this, it may be beneficial to incorporate the patient's personality or profile data (e.g., does this patient tend to share, does this patient tend to complain, past personality survey results, etc.).

### Processor 106

The computing device 104 may be configured to process, for example, audio data of a conversation between a patient and an agent to predict a mental condition of the patient. The computing device 104 may optionally be configured to detect the role (e.g., patient or agent) of each speaker in a conversation, detect the topics of the conversation, and to score a survey. The computing device 104 may be a server, network appliance, computer expansion module, personal computer, laptop, personal data assistant, cellular telephone, smartphone device, UMPC tablets, video display terminal, electronic reading device, wireless hypermedia device, or any other computing device capable of being configured to carry out the methods described herein. Computing device 104 includes at least one processor 106, at least one memory 108, and at least one network interface 110.

The processor 106 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof. The processor 106 may include an audio pre-processor 112, a role detector 114, a language processor 116, acoustic processor 118, fusion model 120, and topic detector 122, survey scorer 124.

### Memory 108

The memory 108 may include instructions for carrying out the functions of any one of the audio pre-processor 112, role detector 114, language processor 116, acoustic processor 118, fusion model 120, topic detector 122, survey scorer 124. The memory 108 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

### Network Interface 110

The network interface 110 enables computing device 104 to communicate with other components (e.g., the input device 102 and the output device 126), to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

### Output 126

The output device 126 may be configured to provide an output. For example, in some embodiments, the output device 126 may comprise a display. In some embodiments, the display may be configured to provide an agent with real-time or just-in-time results. In some embodiments, the agent may be capable of accessing results previously processed using the output device 126. In some embodiments, the output device 126 may be configured to provide some or all of the results of the determination of the computing device 104 to, for example, a database for storage and/or a further computing device for further processing or other output reasons. In some embodiments, the output device 126 comprises display screen and a speaker.

Results delivery can include providing results to the agent for use in documenting the conversation and in deciding next steps for the patient with respect to mental health screening or management. For example, the results delivery can include summarizing the conversation, analyzing the conversation, providing predictions and confidence on the predictions and on the data, comparing to the conversation prior data and results etc.

In some embodiments, the results are delivered in real-time (i.e., real-time mode), wherein results can be updated dynamically. In some embodiments, the results are delivered at the end of the conversation, but while the agent still has the patient on the line (i.e., just-in-time mode). In some embodiments, conversations may be processed in batch after they have concluded (i.e., offline mode). In offline mode, results may be delivered centrally or to the agent directly.

In real-time interactive mode, embodiments of the system can enable an agent to dynamically modify the course of the conversation with the patient to achieve better results. For example, the system can let the agent know to insert a mental health relevant question into the existing conversation, if not enough information on the patient's mental health has been yet addressed. The system can also dynamically let the agent know if the system is already confident enough in its prediction so that the agent could save time and wrap up the conversation. The system can also let agents know when to ask for more details or encourage longer turns.

In just-in-time mode the pre-processing may strictly be sequential (i.e., pre-processing is done before you identify the role and then de-identify the information). In just-in-time mode, it may be beneficial to terminate the passive listening slightly in advance of the end of the conversation to give the system time to analyze the conversation. In some embodiments, the system may be configured to predict when the conversation is coming to an end (e.g., based on changes in pitch) so that they just-in-time analysis will finish just as the conversation ends. Such embodiments may provide an automatic just-in-time analysis in that the analysis is carried out without any further input from the agent.

Offline mode may provide a more economical analysis (i.e., running batches of conversations offline when demands for processing power may be lower and can dynamically be adjusted without impacting the final delivery time).

As part of the output provided to the user, the system may provide analytics. Analytics can provide end users with information about the content, quality, and interaction in a conversation. Analytics can include an unlimited range of measures derived from output of the processing steps, in particular using word-based information. For example, the system can provide overall time spoken by each speaker, number of words spoken by each speaker, percentage of speech from the patient, lengths of turns, topic distributions, etc. Analytics can capture interpretable and useful information about a conversation, for example response word counts can be used as a proxy for level of engagement (e.g., conversations with low percentages of patient words are probably not high in rapport). Analytics can be used by case management companies to view trends in aggregate, to understand how different agents perform, to know which questions were asked in which conversations, and so on.

The analytics can be based on the text and the audio (i.e., the acoustic information). Patient responses taking certain shapes may indicate that the agent is good at eliciting fulsome responses from the patient. For the systems and methods described herein (and machine learning predictions specifically), the goal is to elicit mental health related cues. In some embodiments (e.g., for case management) there may be more general interest in knowing whether other information is obtained (e.g., not just mental health related cues) and whether there is patient engagement and rapport.

In some embodiments, the analytics can be provided in real-time during the conversation with the patient. The system may be configured to flag if the conversation is not eliciting sufficient information from the patient or if there are outstanding questions that need to be answered empowering the agent to course correct during the conversation. The models need the patient to talk to perform and the models can work better if the agent is coaxing a lot of speech out that is personal. The system may work best when the patient is giving longer responses that are deeper and more reflective in nature.

In some embodiments, the output may further provide a text summary. The text summary may provide a summary of the discussion provided during the conversation. It may be helpful to provide in an end report by the agent or to jog the agent's memory at a later point. In generating this summary without input from the agent, it may save the agent time (giving them an opportunity to review for accuracy, but not requiring the agent to take detailed notes or generate the summary themselves). The text summary may represent a capability that can be used independently of mental health prediction. Text summary can use large language models (LLMs) to summarize the ASR based output from a case management call.

In some embodiments, the topic of conversation may also be extracted from the conversation as provided as a part of the analytics. In providing the topics discussed during the conversation, the metrics may be more interpretable (an end user can see both what the patient assessment is, and which topics were discussed and flagged by the system).

Some embodiments may further provide the survey scoring (as described above) as part of the output. The system can, for example, track the verbal administration of surveys and keep score via a graphical user interface. In, for example, interactive mode, the agent can use that information to better administer and score such surveys. Note that in interactive mode, the goal may not be to distract the patient, so these improvements will stem from the agent's use of the information (to the patient the conversation proceeds naturally).

### EXAMPLE UI

The systems and methods described herein can offer a call intelligence tool that uses AI to quantify behavioural health issues such as assessing depression and anxiety severity to triage and monitor effectively, minimize administrative tasks with note summarization, analyze different aspects of the call (such as patient engagement, topics discussed, therapeutic alliance, etc.) and provide agent guidance in real time.

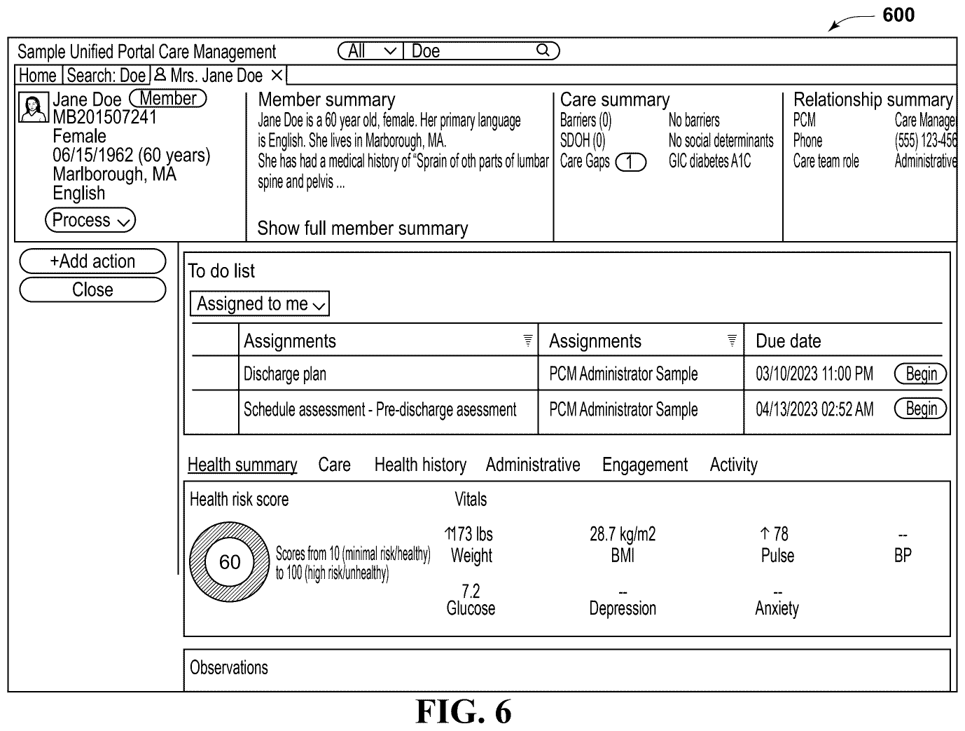

FIG. 6 illustrates an example UI 600 for case managers, according to some embodiments.

In some embodiments the care manager can open the software to launch the example UI. The launch pad can provide information to the care manager such as the patient's name and information, a care summary (e.g., including any barriers to healthcare, social determinants of health, and care gaps). The UI 600 can also include information such as any recent assignments (e.g., discharge plans or scheduling assessments). The UI 600 can also include the patient's health risk score.

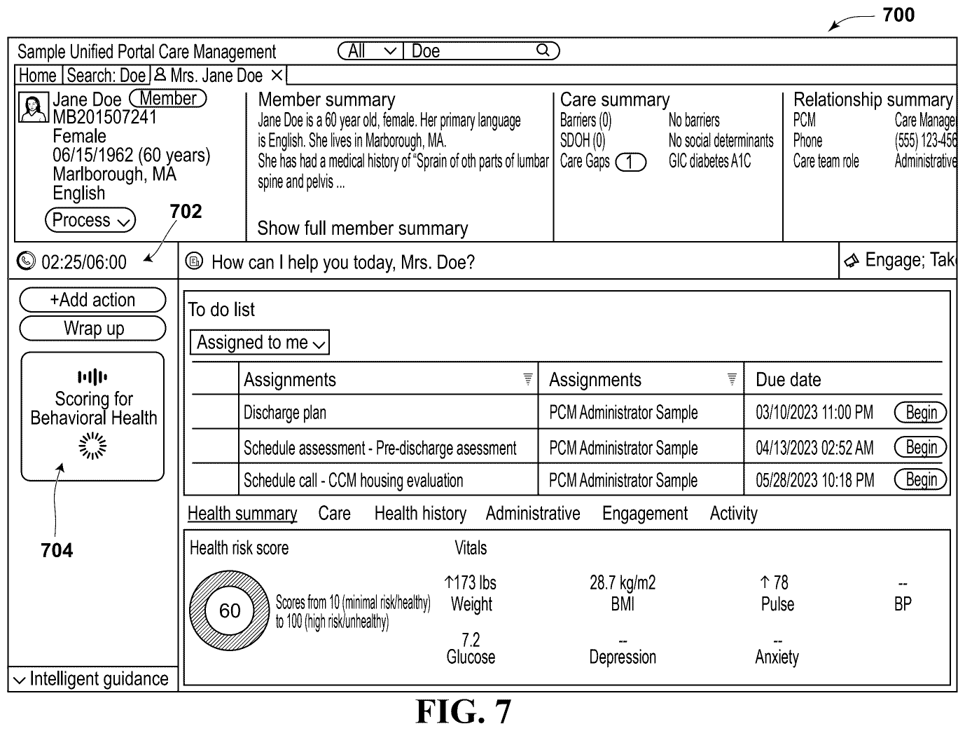

FIG. 7 illustrates an example UI 700 for case managers while calling a patient, according to some embodiments.

From this UI portal, the care manager can call the patient (see icon 702) to begin the workflow. While calling the patient or during the very beginning of the conversation, the system may display a loading screen for the behavioural health score (704).

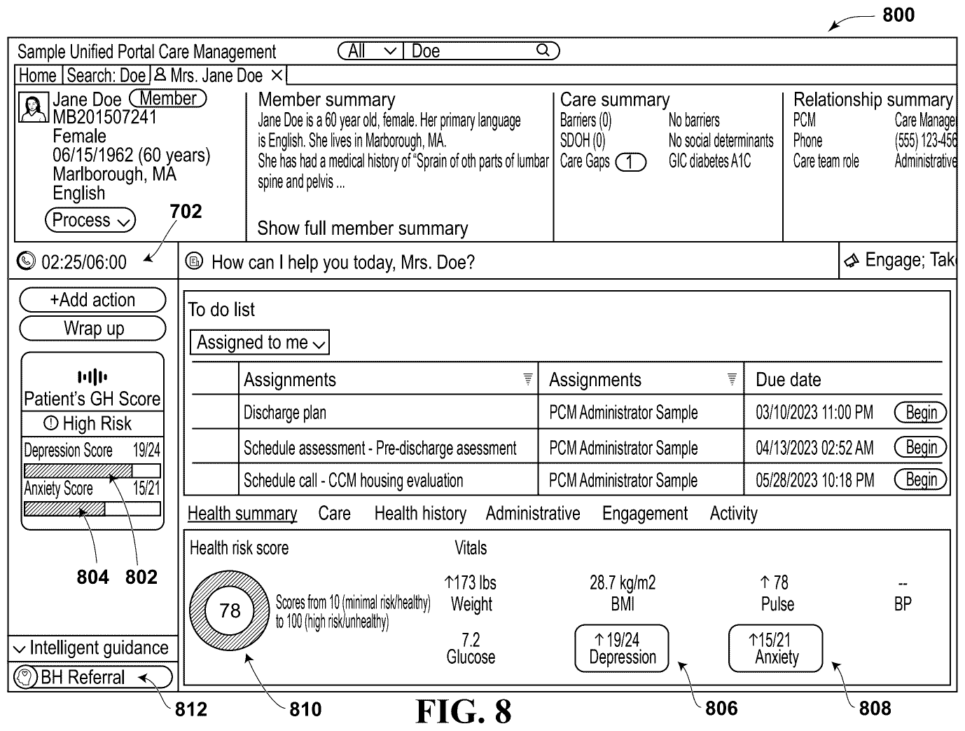

FIG. 8 illustrates an example UI 800 for case managers while the system is delivering a behavioural health score, according to some embodiments.

As the care manager talks to the patient the system can analyze what the patient is saying and how they are saying it to deliver behavioural health scores. For example, the behavioural health scores for depression 802 and anxiety 804 can be displayed. As a further example, this information can be provided with past data to indicate whether the patient's score is increasing or decreasing (see increase in depression 806 and in anxiety 808). Other health scores are conceived such as one for stress.

Using existing care pathways, the system can triage care based on severity and recommend next best actions. For example, the care manager could be prompted to send a behavioural health referral because of the high scores (see the recommendation 812 under intelligent guidance). The care manager can conduct all of these actions during a call to ensure timeliness of care. The behavioural health scores can also factor into health risk scores 810 to provide information about the patient's risk of readmission.

The care manager may also access the call summary from the call for future reference.

Some systems and methods can make screening, monitoring and triaging behavioural health problems seamless and quick ensuring a better patient experience and quality of care while saving time.

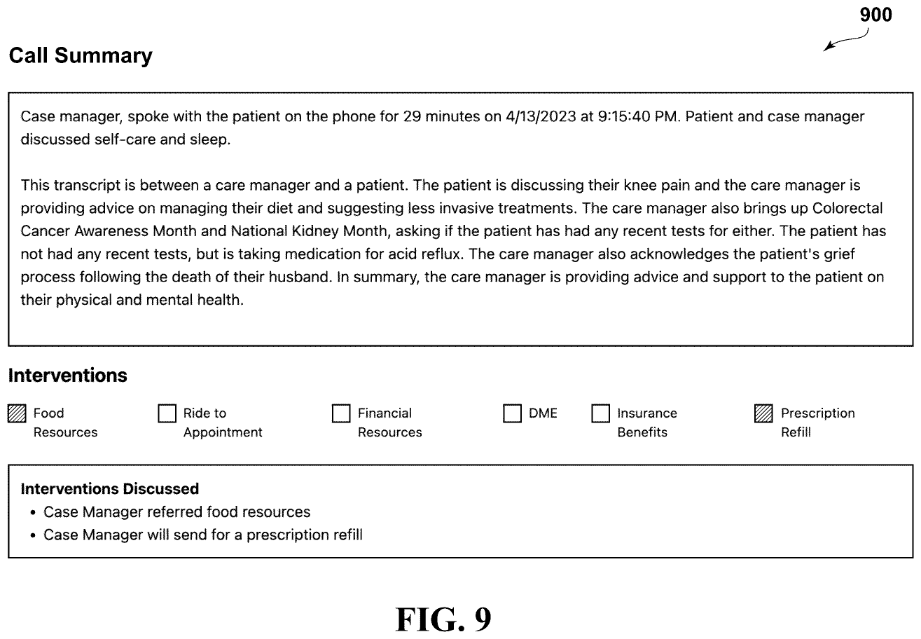

FIG. 9 illustrates an example call summary 900 of a care meeting, according to some embodiments.

In some embodiments, the system may be further configured to generate notes summaries based on a care meeting between the patient and the care manager. The call summary may include logistic information about the meeting (e.g., when the meeting took place, for how long, etc.). The call summary may also include a summary of the topics discussed during the meeting. These summary notes may be pulled from the language model output based on machine learning algorithms. The call summary may also include the interventions that were discussed during the meeting along with relevant details related to those particular interventions. The interventions may be pulled out using, for example, machine learning algorithms applied in the language or acoustic models.

### General System Operation

At runtime, systems and methods described herein may conduct data preprocessing, model inference (severity of symptoms of mental health condition prediction, which may include analytics, text summary, and/or survey scoring), and results delivery.

The systems and methods described herein may use one or both of acoustic and language models and optionally their fusion. In some embodiments, all available models for health conditions may be used (e.g., depression, anxiety, etc.). In some embodiments, past information about the patient may be used to analyze longitudinal trends. In some embodiments, metadata about the patient may be used to condition model predictions. In some embodiments, the analytics may produce information on topics discussed, lengths of speech regions, overall speech share by participants and many other analytics. Summarization may be used to produce automatic summarization of the conversation. The systems and methods described herein may be configured to handle multiple languages (and may potentially include code switching). Topic regions may be determined in the conversation and those regions may be weighted dynamically for more efficient processing and higher accuracy of models. Such weightings may be used as an explanation of where cues lie. Data from the agents (in addition to the patient) may be used for language processing. The language data may be used to find speech regions on which to run the acoustic model. Roles of the speakers (e.g., patient or agent) may be automatically detected. Confidence scoring may be used to measure, for example, quality of the signal (e.g., SNR clipping), confidence from third-party ASR, length measures, and model-based estimates.

### Pre-Processing

In some embodiments, the input data may be pre-processed. For example, audio data may be processed by an audio pre-processor 112. Other pre-processing modalities are also conceived (textual pre-processing, visual pre-processing, etc.).

The audio pre-processor 112 may be configured to optionally process any audio data received from the input device 102. In some embodiments, the audio signals may be pre-processed to filter out irrelevant noise (e.g., through the use of bandpass filters) or to render the data into a format usable by computing device 104.

Deidentification can take place prior to audio or language processing. The recording can be run through Automatic Speech Recognition (ASR), the Protected Health Information (PHI)/Personally Identifiable Information (PII) is marked, then (white noise or some other variant to either mask or remove the audio content) is placed in the PHI/PII regions for audio processing and the PHI/PII regions are removed for language processing.

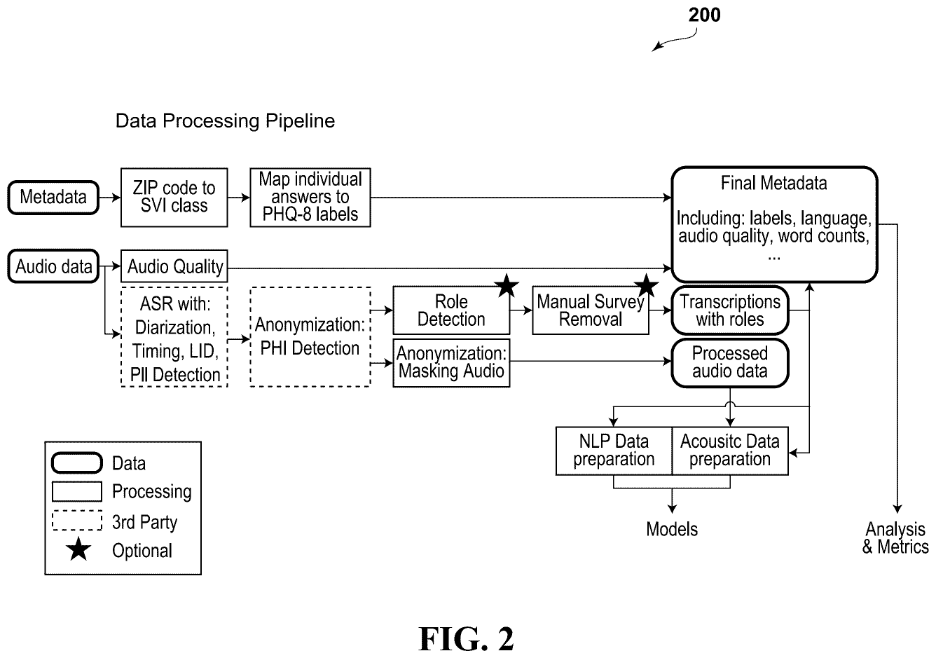

FIG. 2 illustrates a method 200 of pre-processing the conversation data, according to some embodiments.

In some embodiments, the metadata and audio data (e.g., conversation data) may be processed in parallel.

For example, some systems may determine information about patient demographics that relate to mental health conditions. For example, zip code can be mapped to an SVI class (Social Vulnerability Index class). This information may then be used to map to individual answers in, for example, the PHQ-8 labels. The audio data may have its audio quality determined. All of this information may be included as final metadata of the meeting.

The audio data may also have ASR applied to obtain words from the audio. In some embodiments, the system may also provide diarization of the meeting, timing, Language ID (LID), PII Detection, etc. The data may be anonymized to detect any PHI and anonymize. This processed information may then be split into the transcription information which may assess the transcriptions for, for example, Role Detection (as described in greater detail below) and Manual Survey Removal (this may be done so that the model can be trained based on non-survey conversations rather than simply relying on a patient's response to survey questions) and the audio data which may be anonymized by providing masking audio. In some embodiments, the ASR may be bundled with other services such as diarization, PHI and PII detection, or LID detection.

Audio masking may include applying white noise or some other form of audio masking. In some embodiments, the PII and/or PHI may be masked with silence or other audio distortions. It may be important to train the system to handle such silence or audio distortions. In some embodiments, the PII and/or PHI detection may require specific trusted PII/PHI detection software (e.g., to comply with regulations) in use. Such software may be computationally expensive and/or only accessible for a fee. In some embodiments, it may be advantageous to train the system with PII/PHI detection software which may mimic the specific trusted software, but that may be computationally more straightforward and/or more affordable. Such software may be usable during training where the PII and/or PHI will not be seen by other entities (e.g., the data will be used entirely in house where access is limited) but not in actual use where the session information may be distributed to external entities. Such embodiments may be trained on configurations that more or less match the use of PII/PHI detection and masking but are trained on cheaper (financially and/or computationally) software. Such embodiments may be advantageous to train practical models without incurring the potentially higher overhead required for the trusted models.

The conversation transcriptions (with roles) and the processed audio data may then be input into the acoustic data preparation (for subsequent model analysis, e.g., acoustic model analysis). The transcriptions (with roles) may also be input into the NLP (or any other language model) data preparation (for subsequent model analysis, e.g., language model analysis). The final metadata may also be passed through the system for analysis and metrics.

At runtime, data can be run and assigned severity of mental health condition symptom scores for each mental health condition, by trained models (e.g., housed in language processor 116 and acoustic processor 118) and optional model fusion 120. Model output can optionally include measures of confidence (of the data sample audio recording quality, data length, topic content, ASR quality, and model quality among others) to allow end users to decide how much to trust the model predictions on the particular data sample. Confidence can allow the end user to decide to what extent to trust predictions and analytics. ASR confidence is also important for trust in summarization and analytics output (largely based on words).

In some embodiments, inputs required for one component of the system may also be required for another component of the system. By processing information in a manner that reuses these inputs rather than regenerating them, the system may improve its processing economy. For example, models that use ASR outputs (whether words or word time marks) and other types of audio processing may be configured such that the speech recognition process takes a first pass through the data in order to determine, for example, time marks for words. Other audio processing components may also use those time marks for their processes rather than undertaking another pass through the information to regenerate these time marks. For example, the speech recognizer may record the time of each word used by the patient and the audio processor may take the time of the first word used by the patient (potentially with a buffer beforehand) to run audio processing on the strings of patient speech.

According to an aspect, there is provided system 100 for pre-processing transcript data for use in predicting a severity of at least one symptom of a behavioral or mental health condition of a subject. The system 100 comprising at least one input device 102 for receiving conversation data from the subject and at least one computing device 104 in communication with the at least one input device. The at least one computing device 104 is configured to receive in-context learning comprising a description of the behavioral or mental health condition, receive the conversation data from the at least one input device 102, pre-process the conversation data by performing at least of weighing at least one segment of the conversation data based on a relation between the at least one segment of the conversation data and the behavioral or mental health condition, summarizing the at least one segment of the conversation data, providing analytics on the at least one segment of the conversation data, summarizing at least one aspect of the behavioral or mental health condition, and providing analytics on the at least one aspect of the behavioral or mental health condition, and transmit the pre-processed conversation data to one or more models to predict the behavioral or mental health condition of the subject.

In some embodiments, the computing device 104 accesses a large language model to pre-process the conversation data.

### Role Detection

The role detector 114 may be configured to optionally process the data to determine the role of each of the speakers. In some embodiments it may not be apparent what role each speaker is participating as (e.g., agent or patient). Not all set ups for passive listening provide information on which speaker is the patient and which is the agent (e.g., set ups using only one channel for both the patient and the agent). If necessary, role detection can be performed to detect which speaker is the patient. The system may then attribute the audio data to the patient (for acoustic analysis) and attribute the NLP data to the patient and the agent. In some embodiments the agent acoustics may be retained and/or used. Further, some systems may not have a reliable means to discern between the two roles.

The relevant role may generally be the patient or member role so that the systems and methods described herein can be used to assess the patient or member's mental health.

In some embodiments with two or more speakers on the same channel, it may be necessary to identify which speaker is the patient and which is the agent. Doing so will help to map the NLP data onto the correct speaker (a process called diarization). Once the roles are identified, then the system may make use of turn taking to determine which speaker is presently speaking.

Role detection may occur within the first few second or minutes of a conversation so that the system can then provide a real-time assessment of the patient's mental condition based on the conversation. In some embodiments, the role may be detected by detecting the voice of the agent or the patient as compared to prior recordings. For example, the agents may provide sufficient speech samples to identify their voices stored in the system. In some embodiments, the agents may enroll as a speaker in the system so that the system can recognize when their voice is present. In these embodiments, the system may then have a known speaker model for that person. Furthermore, patients may be identifiable if they participate with the system multiple times (and the system takes their speech data in for identification). In some embodiments, the role may be detected by applying machine learning systems to the conversation that are configured to recognize speech patterns (e.g., specific introductory comments such as “how are you”, or intonation) that generally correspond to an agent and assigning that speaker as the agent.

In some embodiments (e.g., for mono audio channels), role detection and diarization can be carried out using ASR. For example, the ASR may be implemented to determine the words and phrases used by each speaker and assign the role of, for example, agent and patient, using the words and phrases used. In some embodiments, the role detection analysis may be carried out using algorithmic and/or machine learning methods. For example, the words and phrases used may be classified using, for example, term frequency—inverse document frequency analysis with hyper parameters tuned with, for example, Bayesian optimization. The Role Detector 114 may be, for example, trained to identify roles of speakers by the use of certain words or phrases. In some embodiments, the role detector 114 may take other factors into consideration as well. In some embodiments, the role detector 114 may be able to call on historic data from one or more of the patients and the agent to aide in its determination (e.g., prior speech samples from the agent stored in, for example, an agent profile).

In some embodiments, there may be two channels (or more) such that role detection may be simplified. For example, if the patient speaks through one channel and the agent through another, then role detection may be assigned based on, for example, the channel of communication. However, even in such embodiments, role detection may nonetheless be advantageous, for example, if there are multiple people (e.g., 2+) on one line. Such a situation may arise, for example, where the patient is being attended to by a caregiver. In such situations, the patient and caregiver may share a channel of communication (e.g., both can be heard through the same channel, or they are passing, for example, a phone back and forth). In such embodiments, role detection can be used to distinguish the role of the patient from that of the caregiver. Other roles may also be distinguished such as translator/interpreter, background chatter (non-entities to the conversation), etc.

An exemplary method for role detection may include running diarization to get the number of speakers & words assigned to each speaker. When two or more speakers have been detected by diarization, then the system may use role detection to assess which speaker fulfills which role if there is no other way to determine role (e.g., if the speakers are not on separate and known channels). For example, the system may score each speaker's words with a role detection model. For example, for each speaker model the log probabilities can be provided for being the Patient or the Agent. For example:
Speaker 1: (−0.1797, ‘[s1]’, ‘P’), (−1.8046, ‘[s1]’, ‘A’),
Speaker 2: (−0.3669, ‘[s2]’, ‘P’), (−1.1804, ‘[s2]’, ‘A’),
Speaker 3: (−1.6420, ‘[s3]’, ‘P’), (−0.2151, ‘[s3]’, ‘A’).

All detected roles can be sorted by probability:
(−0.1797, ‘[s1]’, ‘P’),
(−0.2151, ‘[s3]’, ‘A’),
(−0.3669, ‘[s2]’, ‘P’),
(−1.1804, ‘[s2]’, ‘A’),
(−1.6420, ‘[s3]’, ‘P’),
(−1.8046, ‘[s1]’, ‘A’).

The system may then assign all roles starting from the most highest probability:
Patient: s1
Agent: s3
Other: s2.

When the system detects only one speaker, then this may represent a failure in diarization. The system may use fallback procedures to extract usable information from the session despite the diarization failure.

In some embodiments, the systems described herein may be able to generally identify calls that should be rejected (e.g., IVR—described in greater detail below, family member answers and patient does not join, etc.). Some such systems may, for example, identify that an IVR system has activated. Some systems may identify the conversation data consistent with a family member picking up and the patient not joining (e.g., shorter call, negative response to whether the patient can join, etc.). Such system may improve the trust and performance of the model because inappropriate content is not scored.

In some embodiments, it may be necessary to identify the role of each of the speakers (e.g., agent or patient) before or during analysis. Identification may be important to ensure that the correct conversation data is analyzed (i.e., the patient's conversation data receives focus in the analysis).

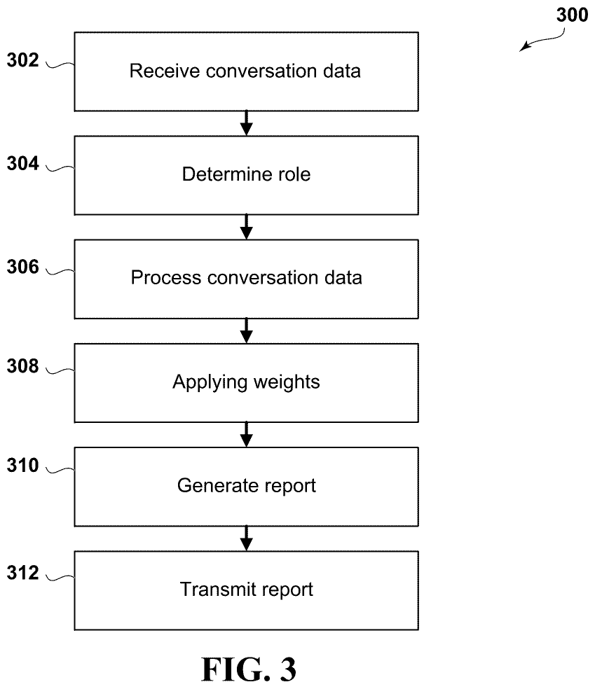

FIG. 3 illustrates a method 300 of detecting roles of speakers, according to some embodiments.

According to an aspect there is provided a method for identifying roles of speakers in a conversation. The method 300 including receiving conversation data from at least one input device (302), determining at least one role of at least one speaker (304), processing the conversation data to generate a language model output and/or an acoustic model output (306), applying weights to the language model output and/or the acoustic model output (308), wherein the language model output and the acoustic model output each comprise a plurality of outputs corresponding to a plurality of time segments of the conversation data, wherein the weights are optionally temporally-based, and wherein the weights are based in part on the at least one role of the at least one speaker during each time segment, generating an electronic report (310), and transmitting the electronic report to an output device (312).

In some embodiments, the method 300 includes fusing the weighted language model output and the acoustic model output generating a composite model. The composite output may represent the fused output from the fusion of the language model output and the acoustic model output.

In some embodiments, the electronic report identifies a severity of at least one symptom of a condition based on the composite output.

In some embodiments, the condition comprises a mental health condition.

In some embodiments, the electronic report comprises an annotation of the language model output indicating salience of at least one of the one or more time segments.

In some embodiments, the at least one speaker comprises an agent and applying the weights to the language model output and the acoustic model output comprises applying a zero weight to acoustic model output corresponding to the agent. In such embodiments, the language model may only use the patient's language information, or the patient plus the agent's language information.

In some embodiments, the weights are based in part on a topic during each time segment.

In some embodiments, the language model output comprises an identification of at least one query based on conversation data from an agent and at least one response to the at least one query based on the conversation data from a patient, wherein the at least one query is mapped onto a predefined query and the at least one response to the at least one query is mapped onto a predefined response to the predefined query.

In some embodiments, processing the conversation data to generate the language model output and the acoustic model output comprises using a language model neural network and an acoustic neural network trained on labelled conversation data collected from one or more other subjects, wherein the labelled conversation data comprises is labelled as (i) having, to some level, a condition and (ii) not having the condition.

In some embodiments, the at least one query is of a set of queries and the computing device is configured to predict an overall score based on the set of queries based on responses to each of the queries of the set of queries.

In some embodiments, the conversation data is processed based in part on at least one of a patient profile and an agent profile, wherein the profile comprises at least one of historical, biographical, demographic, and longitudinal data.

In some embodiments, the conversation data comprises at least one of speech data and text-based data.

In some embodiments, the method 300 is configured to run a model based on human-interpretable features.

According to an aspect, there is provided a non-transient computer readable medium containing program instructions for causing a computer to perform any of the above methods.

### Interactive Voice Response

In some embodiments, the system may be configured to manage Interactive Voice Response (IVR) systems.

IVR technology can allow users to interact with computer-operated telephone system through voice or dual-tone multi-frequency inputs. IVR systems often make use pre-recorded or dynamically generated audio to communicate with a user. The system described herein may encounter such IVR systems, for example, for a voice mail service, to navigate a call routing system, or if a wrong number is dialed. IVR systems may impact the model. For example, the system may attempt to diarize and catalog correspondence with an IVR system, or it may attempt to base a patient's score in part on audio from the IVR system.

In some embodiments, the system is configured to recognize audio arising from an IVR system and remove it or otherwise omit it. In some embodiments, the system has been trained to recognize IVR systems and disregard such audio as appropriate. In some embodiments, the system may disregard IVR system audio from diarization or include it as a special “IVR” speaker (unlike a human speaker). In some embodiments, the system is configured to disregard IVR audio from scoring the patient.

In some embodiments, the system may be configured to identify IVR speakers within the diarization. In some embodiments, the content of the IVR speaker may be used to assist in the prediction. For example, the IVR audio may be of use for scoring a patient's condition if the IVR system is, for example, a call triaging system for a mental health clinic. In such embodiments, the system may be configured to use the language used by the IVR system within, for example, a NLP to assess the language (or dual-tone multi-frequency inputs from the patient on their phone) that the patient uses in order to score the user (e.g., pull the context of the call).

In some embodiments, IVR systems may be recognized based on the words and phrases used common to IVR systems (e.g., “press 1 for . . . ”, “please hang up and try your call again”, etc.). In some embodiments, IVR systems may be recognized by the particular audio patterns that they exhibit (e.g., where there is a patient speaker and an IVR speaker, the system may identify the monotonic, professional, and vaguely cheery speaker as the IVR speaker). In some embodiments, the system may be trained on sample IVR systems to quickly identify the IVR systems and manage that data accordingly.

In some embodiments, the systems described herein may be able to generally identify calls that should be rejected (e.g., IVR, family member answers and patient does not joint, etc.). Some such systems may, for example, identify that an IVR system has activated. Some systems may identify the conversation data consistent with a family member picking up and the patient not joining (e.g., shorter call, negative response to whether the patient can join, etc.). Such system may improve the trust and performance of the model because inappropriate content is not scored.

Once roles are assigned within the beginning of a conversation, then the system may use turn taking to differently analyze data from each speaker. Language models may analyze patient speech more closely. Acoustic models may, for example, weigh the patient speech, more highly and analyze it with more sophisticated models.

The NLP model (described in greater detail below) may or may not use turns but turns can be beneficial to the acoustic model to identify which person is speaking when (and thus weigh patient speech more heavily). The agent is generally used as a starting point (e.g., after the agent's role has been identified by the system) and then system then annotates the data as being between the agent and the patient. The system may annotate for other speakers as well should they be present (e.g., a caretaker and an interpreter).

### Topic Detection

The topic detector 122 may be configured to optionally process the data to determine the topics of conversation. The topic detector 122 may access the general topic of segments of the conversation for processing purposes or assessment purposes. For example, topics may be assessed to determine which segments of the conversation merit closer inspection (i.e., more robust analysis) or higher importance (i.e., higher weightings).

In some embodiments, the system may use information related to detected topics for estimating scoring confidence. In some embodiments, heuristics may be used based on the number and kind of topics and their duration (or number of words) or regions related to each topic. For example, the system may be configured to score the confidence of an output more highly if the topics mentioned in a scored session are the same as the topics encountered during training.

These topic statistics can be also used to improve model training and scoring. In some embodiments, results of topic detection can be provided as an input feature for the models. In some embodiments, regions corresponding to certain topics can be de-weighted or removed entirely from scoring as they may not be as helpful in scoring the session.

Some embodiments may provide some explainability of the prognosis provided by the system by highlighting and flagging relevant passages, expressions, or words from the conversation that were important to the suggested assessment by the system.

Conversations between patient and the agent can be long (thirty minutes to an hour or more is not unusual) and can be full of information that is less relevant for severity of symptoms of mental health condition prediction. The systems and methods described herein can find the most salient regions of the conversation and use it for prediction (especially for the language model) for cost effectiveness as well as accuracy. Automatic detection of topics can be used for this purpose. Topics can be detected in either the speech of the agent, the patient, or both (e.g., the patient response may require an understanding of the agent query for context). Topics relevant for mental health and related states (for example social determinants of health (SDOH)) can be assembled using multiple methods (empirical, clinical, rule-based). Regions containing the estimated relevant topics are given optional higher weight in the modeling, to improve performance. For example, regions of the conversation related to medicine, exercise, living situation (e.g., living alone), interpersonal relations (e.g., family and friends) may be more important (and thus can be weighted more highly) than other portions of the conversation. Further, other salient content that correlates to prediction accuracy may be included and weighted more highly. Regions relating to the same topic may be assessed to determine whether there are many negative, positive, or neutral words (e.g., negative words associated with family may indicate poor familial support which may lend towards prediction of a mental health condition).

In some embodiments, the positive/negative value of a word may be determined from modelling rather than from sentiment of the word. For example, “tennis” may be predictive of patients who are not depressed while “bed” may be predictive of patients who are.

Salient topic regions can also be made available to the system to optimize processing efficiency. Once a portion of the conversation is identified as relating to a salient topic, then it may be beneficial to run more expensive speech recognition on that portion. Third-party automatic speech recognition (ASR) can be expensive and reducing the amount of conversation that will be processed using this ASR can be highly beneficial to the cost of these systems. ASR may be necessary in order to find topic regions themselves, but once found, these regions can be prioritized for the running of higher cost and higher accuracy ASR, for better predictive modeling.

Topic assessment may require an analysis of both the language data from the patient as well as the agent. Though the patient responses are the interesting part of the data, the agent queries (or remarks) may be assessed to ascertain the context of the patient responses. For example, the response “Yes” means very different things if the questions were “Can you hear me?”or “Are you having suicidal thoughts?”.

In some embodiments, the system may employ a computationally lighter model to find cues that may include relevant topics. For example, the system may analyze the session for clusters of words or phrases that are relevant to topics of interest or determine where the patient's voice has a marked difference in emotional expression. The model may be trained to further analyze the key regions in the session differently than the rest of the session (e.g., by using more robust analysis models). Advantages of such a system include increasing efficiency and reducing noise in the data. Language models in particular use history of the session resulting in what can be exponential growth of computational complexity with length of the session. Omitting some portions of the session can save on computational resources. Further these models may cost money to use and so using them on subsets of the sessions (rather than the whole session) may save on financial cost while possibly promoting model efficiency.

In some embodiments, it may be beneficial to identify topics of the conversation. This may enable the system to focus on or further process (i.e., with computationally more robust models) segments of the conversation dealing with important topics such as medicine, exercise, or interpersonal relationships.

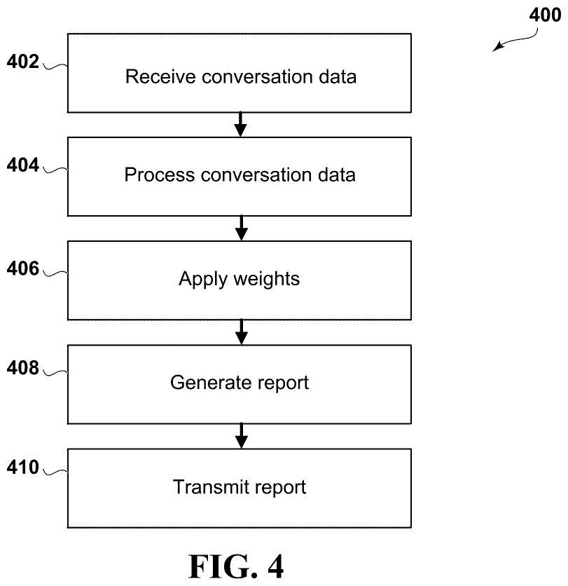

FIG. 4 illustrates a method 400 of detecting topics within the conversation, according to some embodiments.

According to an aspect, there is provided a method 400 for identifying topics in a conversation. The method includes receiving conversation data from at least one input device (402), processing the conversation data to generate a language model output (404), wherein the language model output comprises one or more topics corresponding to one or more time ranges, applying weights to an output to generate a weighted output (406), wherein the output comprises a plurality of outputs corresponding to a plurality of time segments of the conversation data, wherein the weights are optionally temporally-based, and wherein the weights are based in part on the one or more topics during each time segment, generating an electronic report (408), and transmitting the electronic report to an output device (410).

In some embodiments, the method further comprises processing the conversation data to generate an acoustic model output and fusing the language model output and the acoustic model output by applying weights to the language model output and the acoustic model output and generating a composite output from the weighted outputs, wherein the acoustic model output comprises a plurality of outputs corresponding to a plurality of time segments of the conversation data, wherein the weights are optionally temporally-based, and wherein the weights are based in part on the topic during each time segment.

In some embodiments, time ranges of the conversation data corresponding to a predefined topic are processed to generate the language model output using more computationally robust models than those used for time ranges of the conversation data not corresponding to the predefined topic.

In some embodiments, the electronic report comprises a transcript of the language model output annotated based in part on the weights based in part on the topic during each time segment.

In some embodiments, the electronic report identifies a severity of at least one symptom of a condition based on the language model output.

In some embodiments, the condition comprises a mental health condition.

In some embodiments, the electronic report comprises an annotation of the language model output indicating salience of at least one of the one or more time segments.

In some embodiments, further comprising determining at least one role of at least one speaker and wherein the weights are based in part on the at least one role of the at least one speaker during each time segment.

In some embodiments, the language model output comprises an identification of at least one query based on conversation data from an agent and at least one response to the at least one query based on the conversation data from a patient, wherein the at least one query is mapped onto a predefined query and the at least one response to the at least one query is mapped onto a predefined response to the predefined query.

In some embodiments, processing the conversation data to generate the language model output comprises using a language model neural network trained on labelled conversation data collected from one or more other subjects, wherein the labelled conversation data comprises is labelled as (i) having, to some level, a condition and (ii) not having the condition.

In some embodiments, the at least one query is of a set of queries and the computing device is configured to predict an overall score based on the set of queries based on responses to each of the queries of the set of queries.

In some embodiments, the conversation data is processed based in part on at least one of a patient profile and an agent profile, wherein the profile comprises at least one of historical, biographical, demographic, and longitudinal data.

In some embodiments, the conversation data comprises at least one of speech data and text-based data.

In some embodiments, the method 400 is configured to run a model based on human-interpretable features.

According to an aspect, there is provided a non-transient computer readable medium containing program instructions for causing a computer to perform any of the above methods.

### LLM/Chatbot/ChatGPT Implementations

In some embodiments, the prediction models can incorporate large language models into the prediction models. Large language models have the advantage of not using as much labelled mental health data (which can be rare or expensive to produce). They can also include emotion and sentiment content. The large language models can thus act as a source of information for the predictions. These large language models may be used in various combinations and structures. Mental health predictions can be finetuned or adapted. LLMs can be used as generative models to provide real-time guidance and suggestions to health-care professionals during human-to-human (H2H) conversations, including on what content or tone to use in next turns.

In some embodiments, the results from the large language models can be used directly in fusion with other models. In some embodiments, the results from the large language models can be used in a heuristics or rules based set up, a cascade based set up, as a confidence estimator, or any type of learned model, to produce more accurate final estimates when alone or in combination with the models described herein (e.g., one or more acoustic and/or language models).

In some embodiments, when used as a confidence estimator, the action taken by the system to provide the final information to the user can be modified taking into account the confidence estimator. For example, where the large language model produces an answer that is highly divergent than the more highly trained model, the system may take action (e.g., indicate the results may not be confident, troubleshoot processing of the data, request more input, etc.).

In some embodiments, the chat bot may be a prompt-based model, which asks, for example, a large language model for PHQ (or other mental health condition) severity level, based on the transcription. This answer may provide confidence and justification. This model may ask for estimation answers on individual questions, based on the transcription. These individual answers may have confidence and justification. Such individual answers may be aggregated into a final score.

In some embodiments, the transcription may be processed with, for example, a large language model (e.g., ChatGPT) to locate content areas related to a patient's mental health (possibly weighted by how much it is related). This can be used to emphasize these areas during inference or training. In some embodiments, the transcription may be processed with, for example, a large language model (e.g., ChatGPT) to locate content areas not related to a patient's mental health (possibly weighted by how much it is not related). This can be used to lower importance or skip these areas during inference or training.

In some embodiments, the transcription can be processed with, for example, a large language model (e.g., ChatGPT), to summarize several PHQ or/and GAD aspects (this can be extended with other wider behavioral aspects like stress, life satisfaction, wellness, etc.). For instance, each individual PHQ question includes aspects like: “Little interest or pleasure in doing things?”, “Feeling down, depressed, or hopeless?”, “Trouble falling or staying asleep, or sleeping too much?”, “Feeling tired or having little energy?”, “Poor appetite or overeating?”, etc. Or for each individual PHQ topic like: “interest or pleasure in doing things”, “feeling down, depressed, or hopeless?”, “sleep”, “energy level, being tired”, “eating habits”, etc. So, for example, a large language model (e.g., ChatGPT) can extract and summarize each individual aspect including confidence level (how much evidence found for each individual aspect) and justification.

Such data can be used as input features for our models (for training & inference). This can be used alone or together with existing transcription. Such implementations can be used alone or with existing transcription.

### Data Augmentation at Inference Time

In some embodiments, the system may augment the data from the patient (e.g., modifying the signal for both NLP and acoustic models such as by rephrasing, masking, cutting, replacing words with random ones, introducing ASR errors (similar pronunciation replacement), phase shift, add white noise etc.). Such techniques can enable several inferences for the same data point so a distribution of predictions instead of one prediction can be obtained (or a distribution of distributions if the model can generate a probability score).

At runtime there may be a component that tells each model of interest to generate N different input samples using (e.g., predetermined) augmentation approach(es) for each model. Each model may then run on each augmented test sample and combine the results to provide a better performance estimate. For example, each model may apply the score fusion to the result, create a distribution, and use the distribution to come up with a better performance estimate. That could include presenting the distribution itself or using a point estimate from it with confidences.

In some embodiments, the language data can be augmented through the use of large language models. Such models may be used to generate different ways that a patient or agent might say the same thing (e.g., paraphrasing).

In some embodiments, the acoustic data can be augmented through the use of synthetic speech. Synthetic speech (or other methods) may be used to generate different intonations saying the same or different content.

These may provide data augmentation for the purpose of training the models. Fields such as mental health evaluation may have challenges obtaining sufficient raw data for training (data has privacy constraints, and data needs to be labeled close in time to the collection making it even more challenging).

### Natural Language Processing Models (NLP)

The language processor 116 may be configured to process the audio data to extract transcription and language data from audio data received from the input device 102. The language processor 116 may use an algorithm to assess the language data. The language processor 116 may use a natural language processor (NLP) model or combinations of different or differently parameterized language models. The NLP model may use words as input and not the acoustic signal directly.

The goal of including the NLP model is to help predict trends for NLP models generally in this domain. In some embodiments, for the NLP task, the speech signal may first be transcribed using a publicly available ASR services. High ASR error rates may be tolerated by the NLP model (potentially because of good cue redundancy). The NLP model may be based on a transformer architecture and take advantage of transfer learning from a language modeling task. A DeBERTa pre-trained model can be used. Alternatively, RoBERTA, ALBERT, or BERT models can be used.

DeBERTa may have the advantage of having fewer (435 M) parameters relative to other comparable models, while still providing good performance. This can be useful given the large number of experiments to run. In such embodiments, the DeBERTa model can be pre-trained on, for example, over 80 GB of text data from the following common corpora: Wiki, Books, and OpenWebtext. The input context window can be 512 long and the tokenizer can be trained for 128K tokens.

In such embodiments for finetuning, a predictor head can be attached to the language model and a binary classifier can be trained. All hyperparameters other than the learning rate may be fixed. The learning rate can be set proportionally to the amount of training data for each experiment (grid search approach). Early stopping can be used to avoid extensive runtime utilization.

In some embodiments, the language model can be selected from the group consisting of a sentiment model, a statistical language model, a topic model, a syntactic model, an embedding model, a dialog or discourse model, an emotion or affect model, and a speaker personality model. Examples of NLP algorithms are semantic parsing, sentiment analysis, vector-space semantics, and relation extraction. In some embodiments, the methods described herein may be able to generate an assessment without requiring the presence or intervention of an agent. In other embodiments, the methods described herein may be able to be used to augment or enhance agent-provided assessments or aid an agent in providing an assessment. The assessment may include queries containing subject matter that has been adapted or modified from screening or monitoring methods, such as the PHQ-9 and GAD-7 assessments. The assessment herein may not merely use the questions from such surveys verbatim but may adaptively modify the queries based at least in part on responses from subject patients.

In some embodiments, the language model may be trained using a human-to-device corpora. In some embodiments, the language model may be trained using a human-to-human corpus.

In some embodiments, the language model may not use speaker metadata or any other information outside of the information obtained from processing a recorded conversation. The language model may use modern deep learning architectures. The language model may employ large amounts of data, including out-of-domain data, for model pre-training. The language model may be based on a transformer architecture and take advantage of transfer learning from a language modeling task.

The systems and methods disclosed herein may use natural language processing (NLP) to perform semantic analysis on patient speech utterances. Semantic analysis, as disclosed herein, may refer to analysis of spoken language from patient responses to assessment questions or captured conversations, in order to determine the meaning of the spoken language for the purpose of conducting a mental health screening or monitoring of the patient. The analysis may be of words or phrases and may be configured to account for primary queries or follow-up queries. The analysis may also apply to the speech of the agent. As used herein, the terms “semantic analysis” and “natural language processing (NLP)” may be used interchangeably. Semantic analysis may be used to determine the meanings of utterances by patients, in context. It may also be used to determine topics patients are speaking about.

With respect to a weighted word score, semantic models of language processor 116 can estimate a patient's health state from positive and/or negative content of the patient's speech. The semantic models correlate individual words and phrases to specific health states the semantic models are designed to detect. The system 100 can retrieve all responses to a given question from collected patient data and uses the semantic models to determine correlation of each word of each response to one or more health states. An individual response's weighted word score is the statistical mean of the correlations of the weighted word scores. The system 100 can quantify the quality of the question as a statistical measure of the weighted word scores of the responses, e.g., a statistical mean thereof.

Language processor 116 can include a number of text-based machine learning models to (i) predict depression, anxiety, and perhaps other health states directly from the words spoken by the patient and (ii) model factors that correlate with such health states. Examples of machine learning that models health states directly include sentiment analysis, semantic analysis, language modeling, word/document embeddings and clustering, topic modeling, discourse analysis, syntactic analysis, and dialogue analysis. Models do not need to be constrained to one type of information. A model may contain information for example from both sentiment and topic-based features. Language information includes the score output of specific modules, for example, the score from a sentiment detector trained for sentiment rather than for mental health state. Language information includes that obtained via transfer learning-based systems.

Language processor 116 may store text metadata and modeling dynamics and shares that data with acoustic model 118. Text metadata may include, for example, data identifying, for each word or phrase, parts of speech (syntactic analysis), sentiment analysis, semantic analysis, topic analysis, etc. Modeling dynamics includes data representing components of constituent models of language processor 116. Such components include machine learning features of language processor 116 and other components such as long short-term memory (LSTM) units, gated recurrent units (GRUs), hidden Markov model (HMM), and sequence-to-sequence (seq2seq) translation information.

In some embodiments, a navigator can receive language model outputs and semantically analyze the language results for command language in near real time. Such commands may include statements such as “Can you repeat that?”, “Please speak up”, “I don't want to talk about that”, etc. These types of ‘command’ phrases indicate to the system that an immediate action is being requested by the user.

Language models can use the words modeled by natural language processing. NLP models for a native speaker, versus a second language speaker, may likewise be significantly different. Even between generations, NLP models differ significantly to address differences in slang and other speech nuances. By making models available for individuals at different levels of granularity, the most appropriate model may be applied, thereby greatly increasing classification accuracy by these models.

For model inference on data on languages other than the target language many approaches can be used. First, incoming data can be run through language ID, to make a language determination. Then one or more of many approaches can be used. In some embodiments, the non-target language portions can be skipped or otherwise omitted from language scoring. In some embodiments, the system can translate the incoming data into the target language, using, for example, third-party translation. After that, the original target language models can be used. In some embodiments, models can be created for the new language by translating the training data to the new language and training new models. In some embodiments, a model specific to the language used in the incoming data (e.g., created from training data in that language) can be selected. At runtime the right model for the incoming language can be chosen. In some embodiments, models trained to operate on multiple languages (multilingual models) can be used. Such approaches are described in PCT Pat App No PCT/US2022/015147 (published as WO2022169995A1), incorporated herein by reference.

Model confidence for languages foreign to the training data may be impacted. For example, the foreign language may not be well represented in the training data making it difficult to provide high confidence assessments. Analysis of the word string choices made by the patient may be hampered in systems that translate the patient's speech from a foreign language to the target language.

In some embodiments, a speaker (e.g., the patient or a caregiver) may switch between languages during the session. In such embodiments, it may be desirable for the system to continually monitor the language for changes in the language ID. In embodiments, where there is little discussion in a non-target language, it may be simpler (e.g., computationally expedient) to omit analysis of that section of the speech without negatively impacting the model. However, it may be advantageous, particularly where longer swaths of the session occur in a non-target language, to use one of the other approaches described above (e.g., translate the incoming data, use a language-specific model, or use a multilingual model). The approach taken by the system may vary during the session (e.g., the system may initially ignore non-target language until the amount spoken reaches, for example, a certain duration threshold and/or, for example, the analytical value of the non-target language portions may be higher than the computational overhead to analyze it). Embodiments that can ascertain and adapt to language switching may be advantageous where the speakers switch between languages (e.g., a patient that is able the express certain concepts in the target language and other concepts in a non-target language, a patient and caregiver that speak to the agent in the target language and each other in a non-target language) or where speakers are using different languages (e.g., where a patient speaks in a non-target language that may be their native tongue, but where an interpreter or caregiver is speaking in the target language to the agent).

The systems and methods described herein may work without prior knowledge of the speaker's voice, language, or metadata (i.e., the only input that may be required for the estimation of the severity of symptoms of a mental health condition for that speaker is the audio from the conversation).

In the context of modeling one speaker over time, longitudinal information can be used to better calibrate the prediction results (to know whether severity for the patient is increasing or decreasing).

### Acoustic Models

The acoustic processor 118 may be configured to process the audio data to extract conversation data from audio data received from the input device 102. The acoustic processor 118 may be configured to analyze the specific conversation data of the patient to determine whether they have a health condition. Speech models can use the acoustic information in the signal. Acoustic processor 118 can analyze the audio portion of the audiovisual signal to find patterns associated with various health states, e.g., depression. Associations between acoustic patterns in speech and health are in some cases applicable to different languages without retraining. They may also be retrained on data from that language. Accordingly, acoustic processor 118 may analyze the signal in a language-agnostic fashion. Acoustic processor 118 can use machine learning approaches such as encoder-decoder architecture, convolutional neural networks (CNN), long short-term memory (LSTM) units, hidden Markov models (HMM), etc. for learning high-level representations and for modeling the temporal dynamics of the audiovisual signals. The acoustic model may use the speech signal as input and not the words directly.

The goal of including an acoustic model is to represent how acoustic and signal-based models may behave generally in this domain. In some embodiments, the acoustic model used can be based on an encoder-decoder architecture. Alternatively, models using other deep learning architectures, including long-short term memory (LSTM) and convolutional neural networks (CNNs) can be used. The acoustic model can work in two stages. Speech can first be segmented every 25 seconds. The model can learn a latent representation at the segment level, using filter-bank coefficients as input. Representations can then be fused to make a prediction at the response or session level. Model training can use transfer learning from an automatic speech recognition (ASR) task. The ASR decoder can be discarded after the pre-training stage; the encoder can be finetuned along with the predictor layer.

In such embodiments, the encoder can consist of a CNN followed by layers of LSTM. The predictor layer can use a Recurrent CNN (RCNN). The last layer of the RCNN module can be used as a vector representation of a given audio segment. It can be passed along with other representations of the same session to a fusion module that makes a final prediction for the session. The fusion model can use a max operation on all vectors to obtain an aggregate representation for the session, and then use a multilayer perceptron to make a final prediction.

In some embodiments, the acoustic model can comprise one or more of an acoustic embedding model, a spectral-temporal model, a supervector model, an acoustic affect model, a speaker personality model, an intonation model, a speaking rate model, a pronunciation model, a non-verbal model, or a fluency model.

In some embodiments, the acoustic model may be trained using a human-to-device corpora. In some embodiments, the acoustic model may be trained using a human-To-human corpus.

In some embodiments, the acoustic model may not use speaker metadata or any other information outside of the information obtained from processing a recorded conversation. The acoustic model may not use pre-extracted features such as pitch or energy; the input may instead a raw speech signal. Raw input and advanced modeling may produce better results than approaches using pre-extracted features. The acoustic model may use modern deep learning architectures. The acoustic model may employ large amounts of data, including out-of-domain data, for model pre-training. The acoustic model may use transfer learning from an automatic speech recognition task, followed by finetuning.

In some embodiments, the acoustic model can comprise a wav2vec style model. In some embodiments, the acoustic model can comprise a multilingual acoustic model.

In some embodiments the system may make use of speech recognizers. In some embodiments, the system may make use of hesitation, latency, absence of laughter, or other information in the conversation data. In some embodiments, the system may strictly make use of the verbal acoustics. In some embodiments, filler words may be used as an input. For example, people exhibiting depression may use more filler words and thus an analysis of the filler words may improve the accuracy of the model.

Acoustic processor 118 can store data representing attributes of the audio signal and machine learning features of the acoustic processor 118 as acoustic model metadata and shares that data with language processor 116. The acoustic model metadata may include, for example, data representing a spectrogram of the audiovisual signal of the patient's response. In addition, the acoustic model metadata may include both basic features and high-level feature representations of machine learning features. More basic features may include Mel-frequency cepstral coefficients (MFCCs), and various log filter banks, for example, of acoustic processor 118. High-level feature representations may include, for example, convolutional neural networks (CNNs), autoencoders, variational autoencoders, deep neural networks, and support vector machines of acoustic processor 118. The acoustic model metadata allows language processor 116 to, for example, use acoustic analysis of the audiovisual signal to improve sentiment analysis of words and phrases.

In some embodiments, a particular model of acoustic processor 118 may be generated for classifying acoustic signals as either representing someone who is depressed, or not. The tenor, pitch, and cadence of an audio input may vary significantly between a younger individual versus and elderly individual. As such, specific models may be developed based upon if the patient being screened is younger or elderly. Likewise, women generally have variances in their acoustic signals as compared to men, suggesting that yet another set of acoustic models may be advantageous. It is also apparent that combinational models are desired for a young woman versus an elderly woman, and a young man versus an elderly man. Clearly, as further personalization groupings are generated the possible number of applicable models will increase exponentially.

In some embodiments, conversation data is provided to a high-level feature representor that operates in concert with a temporal dynamics modeler. Influencing the operation of these components is a model conditioner. The high-level feature representor and temporal dynamics modeler also receive raw and higher-level feature extractor outputs, that identify features within the incoming acoustic signals, and feeds them to the models. The high-level feature representor and temporal dynamics modeler generate the acoustic model results, which may be fused into a final result that classifies the health state of the individual and may also be consumed by the other models for conditioning purposes.

The high-level feature representor includes leveraging existing models for frequency, pitch, amplitude and other acoustic features that provide valuable insights into feature classification. A number of off-the-shelf “black-box” algorithms accept acoustic signal inputs and provide a classification of an emotional state with an accompanying degree of accuracy. For example, emotions such as sadness, happiness, anger and surprise are already able to be identified in acoustic samples using existing solutions. Additional emotions such as envy, nervousness, excited-ness, mirth, fear, disgust, trust and anticipation will also be leveraged as they are developed. However, the present systems and methods go further by matching these emotions, strength of the emotion, and confidence in the emotion, to patterns of emotional profiles that signify a particular mental health state. For example, pattern recognition may be trained, based upon patients that are known to be suffering from depression, to identify the emotional state of a respondent that is indicative of depression.

Acoustic processor 118 may, for example, consider pitch/energy, quality/phonation, speaking flow, and articulatory coordination. In some embodiments, the acoustic model uses phonemes. In some embodiments, the acoustic model uses the signal pattern directly (i.e., 2 vec acoustic models).

In regular conversation, backchannels refer to interjections made by one speaker in response to another signifying attention, understanding, sympathy, or agreement. These can include both verbal (“Okay”) and non-verbal (head nodding) responses.

In some embodiments, feature extraction may be carried out on the acoustic data. In some embodiments, modelling on the acoustics may be carried out directly on the distribution of energies across frequency bands or spectral/cepstral representations.

In some embodiments, there can be N labels representing different mental conditions and/or states (e.g., stress, anxiety, and depression). In some embodiments there are three labels (stress, anxiety, and depression) that models may be trained separately on. In some embodiments, the system may output results for all three models. The same model can be trained and optimized for different metrics. Some models may have different natures (e.g., there are different kinds of acoustic models).

When translating an incoming language to a target language the acoustic data may not be translated. When the input language is not the same as the language the system is trained for, it is possible to run the acoustic system either “as-is” or to run a system tubed for a language closest to that language. Such embodiments may still provide suitable information in both the language and acoustic data. In preferred embodiments, new models are generated based on training data from the incoming language. In such embodiments, the nuanced detail of the acoustic data may better be analyzed with training data arising from the same language as the incoming language.

In some embodiments, the model may be penalized for learning speaker information. In training data sets, there may be multiple sessions from the same patients. As such, it may be advantageous to actively penalize the model for learning speaker information. During training, the model may be capable of learning to predict mental health conditions by predicting the speaker identity rather than relying on generalizable information from the conversation data. In some embodiments during training, the model may be asked to predict the speaker ID in addition to a mental health condition but be penalized for doing so successfully. For example, a term may be added into the loss function to make the loss larger if the speaker ID is correct. Other methods of penalizing the model are conceived.

### Fusion of Output of Shorter Time Segments

In some embodiments, an input may be segmented into shorter time regions. For example, the acoustic model may need to do segmentation because of the large amount of information in each segment. As a further example, NLP models can also require or benefit from reduction from long string to regions of interest with key information based on results from lightweight training (e.g., to skip over regions of noise (no information to the language model) but retain long term dependencies).

For such embodiments, scoring by the model may be performed on each region. These scored segments may then need to be fused. Different methods (e.g., maxpool, mean, std, var, etc.) to the fusion give different answers and a combination can be used. Segment fusion can be performed in a way that the segment-based models learns a representation that is less correlated with NLP model outputs, leading to potentially better combined performance.

### Fusion of Outputs from Different Models

The fusion model 120 may be configured to optionally combine outputs from the language processor 116 and acoustic processor 118. Estimates for models of both types can optionally be fused to provide better performance and robustness. The fusion model 120 may be configured to assign human-readable labels to scores. Fusion model 120 may combine the model outputs into a single consolidated classification for the health state. Model weighting may be done using static weights, such as weighting the output of the language processor output more than the acoustic processor output. However, more robust and dynamic weighting methodologies may likewise be applied. For example, weights for a given model output may, in some embodiments, be modified based upon the confidence level of the classification by the model. For example, if the language model output classifies an individual as being not depressed, with a confidence of 0.56 (out of 0.00-1.00), but the acoustic model output renders a depressed classification with a confidence of 0.97, in some cases the weight of the models' outputs may be weighted such that the acoustic model output is provided a greater weight. In some embodiments, the weight of a given model may be linearly scaled by the confidence level, multiplied by a base weight for the model. In yet other embodiments, output weights are temporally based. For example, generally the language model outputs may be afforded a greater weight than other output, however with a video output, when the user isn't speaking, a video output may be afforded a greater weight for that time domain. Likewise, if a video output and an acoustic model output are independently suggesting the person is being nervous and untruthful (frequent gaze shifting, perspiration increased, pitch modulation upward, increased speech rate, etc.) then the weight of the language model output may be minimized, since it is likely the individual is not answering the question truthfully.

In some embodiments, the fusion model may be configured to use confidence to modify the fusion weights (e.g., for the entire conversation or by region of conversation). In some embodiments, the system is configured to compute the confidence in segments or turns in the conversation and combine those in, for example the acoustic model. For example, the system may assess segments or turns for their confidence and only admit for analysis segments or turns which produced a certain confidence level.

In some embodiments, the system may ascertain agent performance and use that to modulate the weights of the fusion model (or other models).

In some embodiments, the system may be configured to handle, for example, diarization failure by adjusting the fusion model. In some diarization failures, the system fails to recognize the patient and the agent, for example, mistaking them both as the same person. In such circumstances, the system may be configured to identify a failure, for example, through the detection of only one speaker (i.e., a diarization failure in this example, as there should be at least two speakers) and to apply custom weights to the fusion model.

The custom weights can be 0 for the acoustic model output and 1 for the language model output. Even in the event of diarization failure, the words of both the patient and agent can be probative into the patient's mental state. The acoustic model may be comparatively less helpful as it will mix the acoustic data from both the patient and the agent. Diarization failures may also arise where the roles of the patient and, for example, a caregiver or an interpreter cannot be distinguished.

In some embodiments, the fusion weights may be adjusted automatically based on extracted information. For example, information such as signal-to-noise, duration, number of words per role, detected topics, estimated gender or age, estimated distance to the microphone, reverberation level, background noise type detected (e.g., car engine v TV/radio talk v TV/radio music) may be used to adjust the fusion weights automatically. For example, based on the detection of information the system may identify an negative effect on the acoustic data causing it to be less probative (e.g., detection of reverberation levels that interfere with the acoustic data), the fusion model may adjust the weights of the language model output to be higher and that of the acoustic model output to be lower than in a situation where this effect was absent. The magnitude of the modification to the fusion weights may be based in part on the magnitude of the effect detected in the extracted information (for example, a quiet car engine sound may provoke little to no adjustment of the fusion weights, while a loud car engine may provoke a decrease in the weighting of the acoustic model output compared to the language model output). In some embodiments, the character of the extracted information may provoke a specific weight modification (e.g., where the number of words spoken by the patient increase above a threshold, the system may focus more acutely on the acoustic data by weighting it more highly as compared to where this threshold is not overcome).

In some embodiments, the fusion weight may depend in part on metadata (e.g., gender, age, patient category, call category, etc.). For example, based on some metadata, the fusion weights may be adjusted (for example in the form of a modifier over the whole session or a dynamic modifier that adjusts based on further variables assessed during the session itself). For example, the metadata may indicate that the patient is older, and the system may adjust the fusion weights to weigh the language model output more highly than if the patient were younger (e.g., as older patients may speak more quietly and thus the acoustic model may produce less reliable results).

After model output fusion and weighting the resulting classification may be combined with features and other user information in order to generate the final results. These results are provided back to the agent for storage and potentially as future training materials, and also to the output device 126 for display.

### Multiple Health Condition Detection

In some embodiments, the systems and method described herein are trained and deployed as models that can assess multiple health conditions at once. In some embodiments, the system may be configured to assess multiple mental health conditions where those conditions are correlated in the population.

Such models may be advantageous because they may have better performance by capturing correlations of conditions in the population. Such models may also be capable of using an assessment of one condition to assist in the determination of a condition of interest (e.g., if the model does not have sufficient training data on the condition of interest to label it accurately). For example, the system may be configured to assess both anxiety and depression even if there was no or limited depression training data. Such models may also be more efficient if multiple condition outputs are desired.

Such system may be more useful in populations where the correlation between the two conditions is similar or the same as that of the training data. For example, anxiety and depression may be highly correlated in younger population while older populations may have a weaker or no correlation between anxiety and depression. As such, a model trained with a high correlation between anxiety and depression may perform better for younger populations.

### Interpretability

In some embodiments, the system may be configured to run models based on human-interpretable features (e.g., pitch for acoustic modelling, use of pronouns for language modelling) in addition to the systems and methods described herein. In some cases, models described herein can outperform models based on human-interpretable features, but by running these human-interpretable models in addition to the models described herein, the system may be able to provide some explainability for the outputs of the model. In some embodiments, interpretable features in models of one type (e.g., an acoustic model, a language model) can be used as justification for a model of a different type.

In some applications, explainability may be desired. For example, certain regulatory regimes require that predictions from models about mental health conditions be justified in some way. By using the human-interpretable based models in addition to the models described herein, the system may be capable of satisfying such requirements. For example, pitch variation (e.g., monotone) and pronoun use (e.g., using personal pronouns a lot) may be used to justify a finding that the patient has depression, though the deep learning model itself is not improved by the inclusion of such features.

In some embodiments, the explainability may also incorporate longitudinal information, for example, from a patient's profile. In such embodiments, the system may not only run analysis on the patient's information in a single session, but how it compares to their conversation data from past sessions. Such analysis may be able to ascertain that the patient is using, for example, more personal pronouns and this may be the provided justification for a higher score on, for example, a depression metric.

### Conditional Estimates

In some embodiments, metadata may be used to customize and/or modify the model predictions. For example, medical records, drug treatment, obesity, family history, and other information can be fed into the model. The model may or may not use the information. For example, the system may use this metadata to modify its predictions or select a different model to analyze the patient with. In some embodiments, the system may not use the metadata unless the model predictions exhibit low confidence (e.g., only used as a conditional estimation) and then in such a situation the system may use the metadata to enhance the confidence of the model prediction.

This conditional estimation can be used as a screening tool. For example, based on metadata from a patient, the model may be modified based on those conditions. In this manner the model may be better able to treat like patients alike and different patients differently. Furthermore, it may provide a convenient way to enable comparison between unalike patients (e.g., because the system can attribute some of the adjustment to this metadata).

In some embodiments conditional estimation can be used to undo differences that arise in changes of context between sessions. For example, the patient's conversation data may be impacted by a variety of factors that are not causally linked with a mental condition (e.g., time of day, people in earshot of the session, whether the patient was in a rush, or was almost in an accident immediately prior to the session). The system can be configured to take these inputs and adjust the predictions made for the patient in that session (e.g., had the patient nearly been in a car accident preceding the session, panic or agitation in the patient's conversation data may be attributable to their reaction to the near-accident and the model predictions can adjust to account for that attribution).

In some embodiments, these conditional estimates can be used in a longitudinal fashion. For example, the system may track the metadata associated with each session for a patient and develop a model to understand how the patient behaves at different times of the day. For example, the model may be able to appreciate that the patient is a night owl and consequently does not respond in as alert or engaged a manner for sessions which occur in the early morning. In this situation, the system may modify the model for sessions which occur in the early morning to attribute at least some of the patient's possible inattentiveness or irritability on the time of day as opposed to the patient's condition. In further embodiments, the system may try to schedule sessions with the patient (or prompt the agent to do so) at times that are historically more useful (e.g., in the case of the night owl, scheduling meetings at the same time to ensure consistent patient context and scheduling meetings later in the day when the patient is more likely to provide fulsome answers with which the system may assess the patient).

### Survey Scoring

The survey scorer 124 may be configured to optionally process the data to score a survey (e.g., the PHQ-9, PHQ-2, GAD-7).

Survey Scoring can score a survey based on the conversation. The surveys may include the PHQ-9, the PHQ-2, and the GAD-7. Survey scoring may be used independent of mental health prediction. The systems and devices described herein may include NLP and rule based specialized detectors trained to find agent questions that map to survey questions. The system may then look at the patient reply, and map that reply to one of the forced choice options (e.g., “not at all”, “sometimes”, “often”, “always”, etc.). In both cases the words may be different than those in the fixed survey, making the problem nontrivial. There may be speech from the patient or agent in between questions, and questions may not be all present, may not be in order, or can be separated in time by large amounts. Tracking the score for the verbal administration of surveys may be something that automation could provide to aid accuracy and efficiency for the case managers. In real-time interactive mode it can also prompt the case manager to ask a forgotten question in the survey and so on.

The systems and methods described herein can dynamically assess casual conversations to automatically populate a survey scorecard as it goes on. This may give agents at-a-glance verification.

In some embodiments, the system is not only configured to score a survey and provide a patient's final score, but the system may be configured to predict the patient's answers to one or more questions. In some embodiments, predicting a patient's response to individual questions can improve the accuracy of the overall scoring. In some embodiments, measuring individual questions can aide in identifying subtypes of mental health conditions that the user may be afflicted with.

In some embodiments, labels used in training can be based on weighted contributions from each of the questions in a set of questions. For example, the system may weigh questions different when scoring a survey depending on how they contributed to the overall score. As discussed, the labels used in training may not be based on perfect date (e.g., some questions get skipped or asked in a misleading manner) and so weighing the questions differently than would be expected in the raw test may lead to better performance by the model or more accurate confidence levels.

In some embodiments, the answers to the different individual questions can be used to assess and/or estimate subtypes of mental health states. For example, this can be used to identify sub-types of treatment or estimate subcategories of severity types. Such analysis may give rise to different treatment suggestions for these patients.

In some embodiments, it may be beneficial to extract survey questions from an agent's speech and survey answers from the patient's speech to quickly and efficiently score a patient on a variety of diagnostic or other surveys. In some embodiments, the system may be configured to assess whether there are outstanding questions that remain to be queried or to prompt the agent to clarify a response if it does not map onto the pre-determined responses. In some embodiments, survey scoring may be done in real time to give the agent a going measure of a patient's score on said surveys.

In some embodiments, models can be trained using generated paraphrase type data using large language models (LLMs; e.g., GPT type models). In some embodiments, the system makes use of transformers in its architecture.

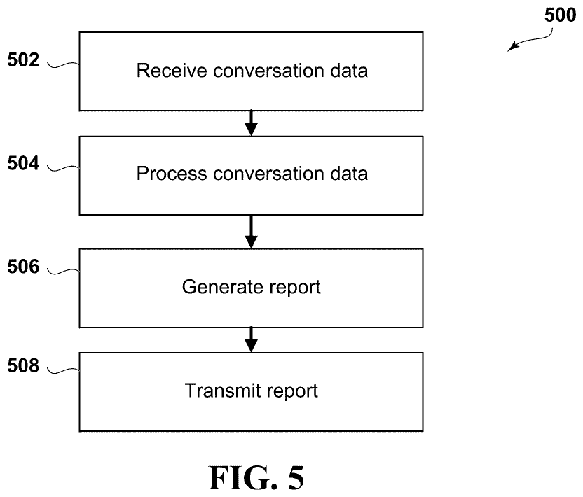

FIG. 5 illustrates a method 500 to score a survey based on passively listening to a conversation, according to some embodiments.

According to an aspect, there is provided a method for scoring surveys based on a conversation. The method includes receiving conversation data from at least one input device (502), processing the conversation data to generate a language model output (504), wherein the language model output comprises an identification of at least one query based on conversation data from an agent and at least one response to the at least one query based on the conversation data from a patient, wherein the at least one query is mapped onto a predefined query and the at least one response to the at least one query is mapped onto a predefined response to the predefined query, generating an electronic report (506), and transmitting the electronic report to an output device (508).

In some embodiments, the method further includes processing the conversation data to generate an acoustic model output, and fusing the language model output and the acoustic model output by applying weights to the language model output and the acoustic model output and generating a composite output from the weighted outputs, wherein the language model output and the acoustic model outputs each comprise a plurality of outputs corresponding to a plurality of time segments of the conversation data, wherein the weights are optionally temporally-based.

In some embodiments, the weights are based in part on the at least one query or a topic during each time segment.

In some embodiments, the method further includes outputting at least one outstanding query on the output device. The outstanding query may be a query to which the user has not provided a response.

In some embodiments, the method further includes outputting a flag indicating that the at least one response to the at least one query is not mapped onto a predefined response to the predefined query with confidence exceeding a threshold.

In some embodiments, the method further includes outputting a prompt to repeat the predefined query when it is determined that the at least one response to the at least one query is not mapped onto a predefined response to the predefined query with confidence exceeding a threshold.

In some embodiments, the electronic report identifies a severity of at least one symptom of a condition based on the language model output.

In some embodiments, the condition comprises a mental health condition.

In some embodiments, the electronic report comprises an annotation of the language model output indicating salience of at least one of the one or more time segments.

In some embodiments, the method further comprises determining at least one role of at least one speaker and wherein the weights are based in part on the at least one role of the at least one speaker during each time segment.

In some embodiments, processing the conversation data to generate the language model output comprises using a language model neural network trained on labelled conversation data collected from one or more other subjects, wherein the labelled conversation data comprises is labelled as (i) having, to some level, a condition and (ii) not having the condition.

In some embodiments, the conversation data is processed based in part on at least one of a patient profile and an agent profile, wherein the profile comprises at least one of historical, biographical, demographic, and longitudinal data.

In some embodiments, the conversation data comprises at least one of speech data and text-based data.

In some embodiments, the method 500 configured to run a model based on human-interpretable features.

According to an aspect, there is provided a non-transient computer readable medium containing program instructions for causing a computer to perform any of the above methods.

### Speaker Profiles

In some embodiments, profiles (or other structures to capture biographical, demographic, historical, longitudinal data or other metadata for speakers) can be implemented to help tailor the model to become more accurate for those speakers (e.g., patients and/or agents) over time. Such embodiments may be configured to assess the patient in an initial session and track patient differences over time to assess their improvement or decline. Patients will naturally vary from one another in terms of their speech and language patterns. An individual may exhibit some characteristics that may suggest a mental health condition while not, in fact having said condition. For example, some patients may exhibit low affect naturally and so may be erroneously categorized as more severely depressed than they actually are. By tracking user differences between session, the system can better assess when the particular patient may be either improving or declining in their mental condition. In this way, the profile may store longitudinal data regarding the speaker. Such information may be stored in a profile. Furthermore, the profile may be able to provide the patient, the agent, or another party with the speaker trends over time (e.g., is the patient improving or declining). The longitudinal data may be usable to predict future risk of a mental health condition for the user based on past patterns of symptom severity over time.

In some embodiments, the patient profile can be built up from patterns in the speech and conversations with the patient. The profile may subsequently be used to predict which therapies and interventions will work best for the patients.

In some embodiments, the system may predict differences between sessions. For example, the system may expect the patient's condition to ameliorate from session to session while that patient is undergoing treatment. This may monitor the patient over time for efficacy of treatment (e.g., treatment type/titration or therapy). Such approaches may be advantageous as they can use a relative score for the particular patient rather than an absolute level to better assess if and when the patient should see a care provider or seek other attention. Patients that deviate significantly from those predictions may cause the system to alert the agent that the treatment option may be ineffective (and may suggest further alternatives), that the patient is not adhering to their treatment regime (e.g., not taking their medication), or something else. As a further example, if the patient has been put on a new therapy which will not be effective for a few sessions, the system may predict that the patient's condition should remain relatively stable until the therapy is expected to kick in. In further embodiments, the systems and methods described herein may be used to longitudinally evaluate a patient's condition and adjust dosage as needed.

In some embodiments, the systems and methods described herein may be used to predict future patient status. For example, for conditions which may involve relapse (e.g., addiction), manic episodes (e.g., bipolar disorder), efficacy of treatment, and efficacy of treatment (e.g., for future treatment). Such embodiments may be useful to predict changes in patient status before they happen and potentially suggest interventions to avoid or mitigate the future condition. For example, in the case of relapse, the system may suggest a greater level of counselling. As a further example, it may alter the treatment or otherwise suggest resources for the patient. As a final example, for treatment which may last for unpredictable amounts of time (e.g., if the patient has first been prescribed it and it's not clear how long it will take to metabolize), the system may monitor the patient to ascertain when the treatment is losing efficacy and suggest repeat treatment. This may be efficient to ensure the patient's condition is treated without being overtreated. Furthermore, the system may be configured to monitor for treatments losing their efficacy with repeated treatments and adjust the treatment (e.g., dosage) with time.

Treatment may include any treatment scheme or other compliance regime. For example, in some embodiments, the treatment is an exercise routine, a nutrition plan, cognitive behavioural therapy, or mindfulness exercises. In some embodiments, the treatment plan is a combination of two or more elements (e.g., a drug and a nutrition plan). Any type of treatment or therapy may be compatible with this system.

In some embodiments, the system may detect when the patient is much worse than a previous session and/or much worse than expected. The system may then alert the agent. This alert can occur early in the conversation so that the agent may steer the conversation to topics that might further aide in eliciting more information or to provide the patient with interventions to improve their condition. This alert may also be used so that the agent or the system can alert another party such as the patient's physician or an emergency medical professional. The patient may be referred to another party by the system or the agent.

In some embodiments, the system may be configured to track a user that has received a treatment that will wear off. The system may be configured to predict when the treatment is likely to wear off (and the user may require a further treatment session). The system may be configured to monitor sessions for cues that the treatment is wearing off and further adjust the trajectory for that patient. As the patient cycles through these treatment trajectories, the system may be able to predict and recommend when next the patient needs treatment. Furthermore, by using the longitudinal data, the system may be able to increase its accuracy with the same patient over time or to assess whether the treatment is becoming less effective with time.

In some embodiments, the speakers may initially be asked to participate in actions that result in a personalized profile (e.g., calibrate a profile). For example, the system may ask them to respond to a series of prompts to ascertain a baseline for the speaker. The system may also be configured to update the profile, based on speaker sessions, to further tailor the profile to the user.

In some embodiments, the patient profiles may store data about the patient. For example, the profile may store information about the patient's introversion and extraversion, their tendency to talk and/or listen, their agreeableness, etc. Such metrics may further be used by the system to match the patient with an agent that the system may predict may more successfully build rapport with the patient.

In some embodiments, the system may be configured to assess a “speaker type” for the patient. Speaker types may include, for example, an indication for the cues that might present in the speech that may indicate specific severity of symptoms of mental health conditions. Such types may aide the system in reducing variability in severity predictions.

In some embodiments, the system may ascertain metadata information during session with the patient. For example, the agent may ask the patient questions during the session and the responses may be incorporated as metadata or the system may make inferences based on conversation data from the patient (e.g., determining that a patient has a history of smoking based on their voice signal). In some embodiments, the system may seek to confirm such inferences (e.g., prompting the agent to ask or the user to respond). In some embodiments, conversation histories (including those of the patient, those of the provider, and those of patient/provider dyads) can be used to analyze how to most efficiently spend time in future conversations, to triage patients, and to adjust the matching of patients and providers. Current and past conversations can be used to discover subcategories of mental health conditions by patient. These subcategories can be used to more effectively design treatment.

End users include not only the agents but administrators, supervisors, and leadership. In some embodiments, the agents may also have profiles. These profiles may be used to assess the agent's performance over time. These profiles may also be used to assess the agent's relationship with specific patients with time. For example, the system may find that the agent-patient relationship follows a certain trajectory of rapport-building if it is a good match. The system may preferentially match agents with patients that they are likely to build rapport with (e.g., patients similar to other patients that the agent has successfully built rapport with in the past).

Agents may not all conduct patient interviews equally well. For example, agents can also influence the patient's answer with leading comments. For example, an agent may ask the patient whether they have been feeling suicidal as “Oh, you don't feel suicidal, do you?” which may lead the patient to answer “No”, consistent with the clear expectation set by the agent's phrasing. Such leading questions may cause certain conditions or symptoms to be over- or under-reported.

The systems and methods described herein may be useful for assessing and monitoring the efficacy of the agents themselves. For example, the manager of agents may be able to see if agents are compliant with policy, regulation, or standards of asking survey or required questions. The managers may also be able to assess whether some agents are better or if the session is better labelled.

The systems and methods described herein may also aid in improving clarity of communication, improving rapport between agent and patient, and inducing compliance with questionnaires and policy.

Rapport may be useful to attract new patients to the case management company. Rapport may also encourage the patients to provide full and honest answers thereby potentially identifying patient issues earlier and obviating the need for a hospital visit (and thus saving money). The systems and methods described herein benefit from conversations where the patient is sharing a lot of information and, in particular, information about their state. Real-time rapport monitoring may help the agent ascertain how much rapport has been built and whether they need to build more. Rapport may map features that correlate to rapport (e.g., small gaps in speech, rate of speech, use of backchannels like “yea”, etc.).

Improving the rapport between the patient and the agent may make the patient more forthcoming. The more forthcoming the patient is, the more input the models have to work with the accurately predict any mental conditions that the user might have.

In some embodiments, the system may assess an agent based on what and how many mental health relevant questions the agent asks. In some embodiments, the system may assess an agent based on how deeply the patient answers questions asked of them (patients more willing to speak freely may have good rapport with their agent). In some embodiments, the system can automatically monitor the agent's performance. In some embodiments, the system may identify areas for improvement for the agent (e.g., instructions to not interrupt the patient, instructions to acknowledge the patient or ask for clarification).

In some embodiments, a parallel diarization model is assessing the agent data. For example, the acoustic and language data may be used to determine the agent's performance. In such embodiments, the agent may provide voice samples such that the system can more readily recognize the agent (and perhaps, as a result, the patient as the other speaker by process of elimination). Such embodiments may store the agent information in, for example, an agent profile which may track that agent's longitudinal performance and/or other metrics.

In some embodiments, an agent's performance metrics may feed into the confidence of the session or parts of the session. In human-to-device implementations, factors which may impact confidence include, for example, audio quality, ASR, length, model estimates, etc. (which all may be factors to impact human-to-human implementations). With an agent, the agent's performance may also impact the confidence of the results of the model. Some further factors which may impact model confidence may include, for example, topics and lengths of topics related to mental health. The performance of an agent may affect confidence in results for their overall sessions (e.g., their coverage) and/or with particular patients (e.g., based on rapport). The system may use this performance to ascertain the trust in any labels which were output.

In some embodiments, the agents may be scored on their ability to accurately label a patient based on a standardized test (e.g., PHQ-9, PHQ-2, GAD-7). The system may be configured to semantically analyze a session and score that session against a question set of interest. The system may be configured to detect questions in the session that closely correspond to the questions in the set. The closer the agent asks the questions to the manner in which they are provided in the set, the higher the agent's score. This may be used to assess the trustworthiness of the agent's scoring of the patient based on the standardized test or the trustworthiness of the system's own score for the patient (as the system may be unable to properly score a patient that was not asked all of the questions in the set or not asked them properly).

### Exemplary Aspects

According to an aspect there in provided a system 100 for identifying roles of speakers in a conversation. The system including at least one input device 102 for receiving conversation data from at least one user, at least one output device 126 for outputting an electronic report, and at least one computing device 104 in communication with the at least one input device 102 and the at least one output device 126. The at least one computing device 104 configured to receive the conversation data from the at least one input device 102, determine at least one role of at least one speaker using a role detector 114, process the conversation data to generate a language model output and/or an acoustic model output using language processor 116 and acoustic processor 118 respectively, apply weights to the language model output and/or the acoustic model output, wherein the language model output and the acoustic model outputs each comprise a plurality of outputs corresponding to a plurality of time segments of the conversation data, wherein the weights are optionally temporally-based, and wherein the weights are based in part on the at least one role of the at least one speaker during each time segment, and generate an electronic report, and transmit the electronic report to the output device 126.

In some embodiments, the at least one computing device is further configured to fuse the weighted language model output and the acoustic model output using fusion model 120 generating a composite output. The composite output may represent the fused output from the fusion of the language model output and the acoustic model output.

In some embodiments, the electronic report may identify at least one symptom of a condition based on the composite output.

In some embodiments, the condition may comprise a mental health condition.

In some embodiments, the electronic report comprises an annotation of the language model output indicating salience of at least one of the one or more time segments.

In some embodiments, the at least one speaker comprises an agent and applying the weights to the language model output and the acoustic model output comprises applying a zero weight to acoustic model output corresponding to the agent. In such embodiments, the language model may only use the patient's language information, or the patient plus the agent's language information.

In some embodiments, the weights are based in part on a topic during each time segment.

In some embodiments, the language model output comprises an identification of at least one query based on conversation data from an agent and at least one response to the at least one query based on the conversation data from a patient, wherein the at least one query is mapped onto a predefined query and the at least one response to the at least one query is mapped onto a predefined response to the predefined query.

In some embodiments, processing the conversation data to generate the language model output and the acoustic model output comprises using a language model neural network and an acoustic neural network trained on labelled conversation data collected from one or more other subjects, wherein the labelled conversation data comprises is labelled as (i) having, to some level, a condition and (ii) not having the condition.

In some embodiments, the at least one role of the at least one speaker includes at least one of a patient, an agent, an interactive voice response, and bot speaker.

In some embodiments, the weights applied to the language model output and the acoustic model output are based in part on determining that a number of the at least one speaker matches an expected number of speakers.

In some embodiments, the conversation data is processed based in part on at least one of a patient profile and an agent profile, wherein the profile comprises at least one of historical, biographical, demographic, and longitudinal data.

In some embodiments, the conversation data includes at least one of speech data and text-based data.

In some embodiments, the at least one computing device 104 is configured to run a model based on human-interpretable features.

According to an aspect there is provided a system 100 for identifying topics in a conversation. The system 100 including at least one input device 102 for receiving conversation data from at least one user, at least one output device 126 for outputting an electronic report, at least one computing device 104 in communication with the at least one input device 102 and the at least one output device 126. The at least one computing device 94 configured to receive the conversation data from the at least one input device 102, process the conversation data to generate a language model output using the language processor 116, wherein the language model output comprises one or more topics corresponding to one or more time ranges, apply weights to an output to generate a weighted output, wherein the output comprises a plurality of outputs corresponding to a plurality of time segments of the conversation data, wherein the weights are optionally temporally-based, and wherein the weights are based in part on the one or more topics during each time segment, generate an electronic report, and transmit the electronic report to the output device 126.

In some embodiments, the at least one computing device 94 is configured to process the conversation data to generate an acoustic model output using acoustic processor 118, and fuse the language model output and the acoustic model output using fusion model 120 by applying weights to the language model output and the acoustic model output and generating a composite output from the weighted outputs, wherein the acoustic model output comprises a plurality of outputs corresponding to a plurality of time segments of the conversation data, wherein the weights are optionally temporally-based, and wherein the weights are based in part on the topic during each time segment.

In some embodiments, time ranges of the conversation data corresponding to a predefined topic are processed to generate the language model output using more computationally robust models than those used for time ranges of the conversation data not corresponding to the predefined topic.

In some embodiments, the electronic report comprises a transcript of the language model output annotated based in part on the weights based in part on the topic during each time segment.

In some embodiments, the electronic report identifies a severity of at least one symptom of a condition based on the language model output.

In some embodiments, the condition comprises a mental health condition.

In some embodiments, the electronic report comprises an annotation of the language model output indicating salience of at least one of the one or more time segments.

In some embodiments, the computing device is further configured to determine at least one role of at least one speaker and wherein the weights are based in part on the at least one role of the at least one speaker during each time segment.

In some embodiments, the language model output comprises an identification of at least one query based on conversation data from an agent and at least one response to the at least one query based on the conversation data from a patient, wherein the at least one query is mapped onto a predefined query and the at least one response to the at least one query is mapped onto a predefined response to the predefined query.

In some embodiments, processing the conversation data to generate the language model output comprises using a language model neural network trained on labelled conversation data collected from one or more other subjects, wherein the labelled conversation data comprises is labelled as (i) having, to some level, a condition and (ii) not having the condition.

In some embodiments, the at least one query is of a set of queries and the computing device is configured to predict an overall score based on the set of queries based on responses to each of the queries of the set of queries.

In some embodiments, the conversation data is processed based in part on at least one of a patient profile and an agent profile, wherein the profile comprises at least one of historical, biographical, demographic, and longitudinal data.

In some embodiments, the conversation data comprises at least one of speech data and text-based data.

In some embodiments, the at least one computing device 104 is configured to run a model based on human-interpretable features.

According to an aspect, there is provided a system 100 for scoring surveys based on a conversation. The system 100 includes at least one input device 102 for receiving conversation data from at least one user, at least one output device 126 for outputting an electronic report, at least one computing device 104 in communication with the at least one input device 102 and the at least one output device 126. The at least one computing device 104 configured to receive the conversation data from the at least one input device 102, process the conversation data to generate a language model output using language processor 116, wherein the language model output comprises an identification of at least one query based on conversation data from an agent and at least one response to the at least one query based on the conversation data from a patient, wherein the at least one query is mapped onto a predefined query and the at least one response to the at least one query is mapped onto a predefined response to the predefined query, and generate an electronic report, and transmit the electronic report to the output device 126.

In some embodiments, the at least one computing device 104 is further configured to process the conversation data to generate an acoustic model output using acoustic processor 118 and fuse the language model output and the acoustic model output using fusion model 120 by applying weights to the language model output and the acoustic model output and generating a composite output from the weighted outputs, wherein the language model output and the acoustic model outputs each comprise a plurality of outputs corresponding to a plurality of time segments of the conversation data, wherein the weights are optionally temporally-based.

In some embodiments, the weights are based in part on the at least one query or a topic during each time segment.

In some embodiments, the system is configured to output at least one outstanding query on the output device. The outstanding query may be a query to which the user has not provided a response.

In some embodiments, the system is configured to output a flag indicating that the at least one response to the at least one query is not mapped onto a predefined response to the predefined query with confidence exceeding a threshold.

In some embodiments, the system is configured to output a prompt to repeat the predefined query when it is determined that the at least one response to the at least one query is not mapped onto a predefined response to the predefined query with confidence exceeding a threshold.

In some embodiments, the electronic report identifies a severity of at least one symptom of a condition based on the language model output.

In some embodiments, the condition comprises a mental health condition.

In some embodiments, the electronic report comprises an annotation of the language model output indicating salience of at least one of the one or more time segments.

In some embodiments, the computing device 104 is further configured to determine at least one role of at least one speaker and wherein the weights are based in part on the at least one role of the at least one speaker during each time segment.

In some embodiments, processing the conversation data to generate the language model output comprises using a language model neural network trained on labelled conversation data collected from one or more other subjects, wherein the labelled conversation data comprises is labelled as (i) having, to some level, a condition and (ii) not having the condition.

In some embodiments, the conversation data is processed based in part on at least one of a patient profile and an agent profile, wherein the profile comprises at least one of historical, biographical, demographic, and longitudinal data.

In some embodiments, the conversation data comprises at least one of speech data and text-based data.

In some embodiments, the at least one computing device 104 is configured to run a model based on human-interpretable features.

According to an aspect, there is provided a non-transient computer readable medium containing program instructions for causing a computer to perform any of the above methods.

### Training

Models can be trained using deep learning. Performance can be validated on labeled data from unseen speakers. The language processor, acoustic processor, and fusion model can each be trained separately, together, or first separately, then finetuned together.

Models can be trained using large amounts of data labeled for patient mental health. In these data sets, the treatment of regions that may contain strong hints to the answer depends on the target deployment data. If that data is not expected to contain survey regions for example, survey regions (if present) can be removed from the training data. The models can thus be pushed to learn about cues to mental health when no standard health survey is administered making the software generally applicable and reducing reliance on the administration of surveys.

In some embodiments, the models may be trained before and after removal of any verbally administered survey. If the model is trained before the removal of the verbally administered survey, then model may not perform well if the deployment data do not contain the survey. If the deployment data do contain a survey that is verbally administered, it may be best to train the models including the survey region in the training data. The survey itself may otherwise make prediction of mental health conditions too easy for the model, and the goal is performance without requiring the survey to be present.

As part of training the survey scoring implementation, it may be necessary to provide the model with a plurality of ways of phrasing different questions and different answers.

Training may make use of a further methods that detect and remove portions of a training conversation that relate to any surveys. These survey-removed conversations can then be used to train the models used in the system to identify and assess a patient based on their non-survey responses.

Training models for older populations may require overcoming some unique challenges. Older patients tend to be less technically literate and so it can be important to make the devices as straightforward as possible. The speaking rate and voice volume tends to be slower and softer for older populations requiring potentially specialized training for the modes. Furthermore, older populations may have more health issues that may further impact the recognition of their voice by models.

### Confidence in Training Data

Training data for human-to-human care sessions may not always have trustworthy labels. Generally, for training, the final score on a standardized test as entered by an agent may be used as some or all of the label for the training data. As such, it is important that such standardized tests be carried out completely and accurately to produce a meaningful label. In some circumstances, the agent may ask the questions from the standardized test in an incorrect order, may not ask them all, or may not score the patient's score correctly (e.g., ask the question as a binary then attempt to rate the patient on a graduated scale).

In some embodiments, the system may be capable of assessing the trustworthiness of labels in the training data and, for example, weighing that data accordingly. For example, the system can search the training data to semantically search for questions similar or the same as the questions from a set of interest (e.g., from the standardized test). Sessions which have questions corresponding closely to every question in the set may be more trustworthy than sessions which are either missing some questions or the closest questions asked do not correspond closely with questions from the set (or both). Furthermore, the more questions asked and the more that the agent and patients discuss these the more trustworthy the data may be. Such trust level can be used to filter bad/good data or to weight data during the training. The trustworthiness levels may be used to filter good data from bad data during training. For example, each session may be scored on trustworthiness based on the semantic closeness of questions asked in the session to the questions in the set. The trustworthiness scores may be used to weight the training data and/or include/exclude the training data.

Furthermore, such considerations may equally be applied in use. For example, the agents may be evaluated for the trustworthiness of the labels generated by the system when the agent is asking questions. Such systems can prompt the agent to ask further questions or to do so properly.

### Data Augmentation in Training

In some embodiments, the training data may be augmented to enhance the training of the model prior to deployment. In some embodiments, the language data and the acoustic data can be augmented. Data augmentation may be particularly advantageous to provide training data for demographics that have comparatively little training data available (e.g., data from speakers of a particular dialect or accent, patients with particular speaking styles). Data augmentation may also be useful in adapting the model to specific populations by familiarizing the model to certain types of words and phrases and types of speaking style or voices expected from the target population.

In some embodiments, the language data can be augmented through the use of large language models. Such models may be used to generate different ways that a patient or agent might say the same thing (e.g., paraphrasing).

In some embodiments, the acoustic data can be augmented through the use of synthetic speech. Synthetic speech (or other methods) may be used to generate different voices saying the same or different content. Furthermore, conversation data from unmodified sessions may be modified to capture particular speaking styles.

### Zero-Shot Learning

Zero-shot learning can describe a learning scenario wherein a model is asked to classify subjects from classes that were not observed during learning. In some embodiments, zero-shot learning models may associate auxiliary information with observed and non-observed classes such that the model may be able to discriminate between classes based on some distinguishing features of the subject.

In some embodiments, the models may be trained using some form of zero-shot learning (e.g., wherein one or more classes was not observed during training). Such embodiments may be advantageous because they may obviate the need for at least some labelled training data.

In some embodiments, systems and methods described herein may make use of language models (e.g., large language models) as a form of a zero-shot model. Large language models are models that have been trained (pre-trained, self-supervised learning, semi-supervised learning, etc.) to predict the next token or word based on input text. In some embodiments, such large language models may be used to predict the mental health assessment for a subject and/or the severity of the symptoms.

In some embodiments, in-context learning (e.g., prompt-engineering) may be used to direct a model to predict behavioral or mental health conditions based on zero- or few-shot learning. For example, the large language model may be provided with a description of depression or other condition and from that may be able to predict a subject's severity of depression (and severity level) based on the description and a transcript of the subject's conversation, for example, with an agent.

In some embodiments, zero-shot learning may be used in conjunction with, for example, the language model. In some embodiments, zero-shot learning may be used directly or indirectly. In some embodiments, a large language model can be asked a subject's severity level (e.g., the PHQ risk level or other mental health condition severity level) based on the transcript of a conversation. In some embodiments, a large language model may be asked for a questionnaire (e.g., PHQ or other mental health questionnaire) estimation for the answers to individual questions based on a conversation transcript. In such embodiments, the individual answers may have confidence levels associated with them. The answers may be aggregated into a final score. As a further example, the individual answers may further help subtype a class of the condition. In some embodiments, the outputs of any of the above may further be used as inputs into further models that may not be zero-shot learning models (e.g., non-zero shot fusion models, language models, acoustic models, etc.).

In some embodiments, the output from the zero-shot models may be fused with other models (e.g., with an acoustic model).

In some embodiments, zero-shot learning models may be used to pre-process the data (e.g., as an indirect use). For example, large language models can be used to extract and summarize topics and properties of interest including confidence levels (e.g., how much evidence is found for each individual aspect). Such data may then be used as input features for further models (e.g., for training and/or inference). This can be used alone or together with existing transcription. In some embodiments, the transcription of a conversation can be pre-processed with a large language model to locate content areas related to a patient's mental health (and potentially weighted by how much it is related). This can be used to emphasize (e.g., apply higher weights to) areas of the conversation during inference or training. In some embodiments, the transcription of a conversation can be pre-processed with a large language model to locate content areas not related to a patient's mental health (and potentially weighted by how little it is related). This can be used to deemphasize (e.g., apply lower weights to) areas of the conversation during inference or training. In some embodiments, large language models can be used to locate, provide analytics on, and/or summarize topics in the transcription. In some embodiments, the large language models can be used to locate, provide analytics on, and/or summarize several aspects of a questionnaire (e.g., PHQ or GAD). For example, it may be able to provide individual answers to, for example, PHQ questions like: “Little interest or pleasure in doing things?”, “Feeling down, depressed, or hopeless?”, “Trouble falling or staying asleep, or sleeping too much?”, “Feeling tired or having little energy?”, and “Poor appetite or overeating?” In some embodiments, the large language models can be used to locate, provide analytics on, and/or summarize behavioral aspects (e.g., stress, life satisfaction, or wellness).

In some embodiments, the pre-processed conversation data may be fed into further models. For example, weightings by the zero-shot models may be used by language or acoustic models to analyze the conversation data. As a further example, the topic summaries may be analyzed by language models to predict the severity of symptoms of a behavioral or mental health condition. The results of the pre-processing may also be provided to an end user (e.g., the agent), for example, to assist with explainability.

### Metric- and Distribution-Based Optimization

Different mental health applications may care about different metrics (e.g., binary screening, class-based, regression based, correlation coefficient concordance). Optimal performance on one metric may not guarantee optimal performance on another. Data distributions may also affect metrics differentially.

In some embodiments, the metric of interest and expected approximate target distribution may be selected and the models and/or fusion model may be optimized for that. The fusion weighting can be trained to optimize for the metric of interest. Optimizing for not only the language and acoustic model, but also the fusion model may improve performance of the model.

### Exemplary Training Implementations

According to an aspect, there is provided a system 100 to train a baseline for one or more of a language model, an acoustic model, and a fusion model (the model) to directly or indirectly detect a behavioural or mental health condition using machine learning. The training includes predicting the behavioural or mental health condition in training data using the model, updating the model based on accuracy of the prediction.

In some embodiments, labels in training data are assessed for trustworthiness before or during training and each training datum is reweighed according to the trustworthiness of its label.

In some embodiments, training data is augmented using at least one of paraphrasing or synthetic speech to generate additional training data for use in the training data.

In some embodiments, the training includes predicting a speaker ID and penalizing the model for accurately identifying the speaker ID. In some embodiments, the model will be penalized for learning information about the speaker ID rather than about the speaker's state.

According to an aspect, there is provided a system 100 for predicting a severity of at least one symptom of a behavioral or mental health condition of a subject. The system 100 including at least one input device 102 for receiving conversation data from the subject and at least one computing device 104 in communication with the at least one input device 102. The at least one computing device 104 is configured to receive in-context learning comprising an explanation related to one or more questions of a questionnaire, receive the conversation data from the at least one input device, and predict the severity of the at least one symptom of the behavioral or mental health condition of the subject based on the in-context learning and the conversation data.

In some embodiments, the computing device 104 accesses a large language model to predict the severity of the at least one symptom of the behavioral or mental health condition of the subject based on the in-context learning and the conversation data.

In some embodiments, the prediction of the severity of the at least one symptom of the behavioral or mental health condition comprises a prediction of a result of the questionnaire.

In some embodiments, the prediction of the severity of the at least one symptom of the behavioural or mental health condition comprises a prediction of a result of at least one of the one or more questions of the questionnaire.

### Compatibility

Optionally, the devices, systems, and methods described herein may be used to implement aspect of the devices, systems, and methods in U.S. Pat. No. 10,748,644, titled “Systems and methods for mental health assessment”, filed Sep. 4, 2019, the entirety of which is incorporated by reference herein. Accordingly, the devices, systems, or methods described herein may be interoperable with a method for identifying whether a subject is at risk of having a mental or physiological condition, comprising obtaining data from said subject, said data comprising conversation data and optionally associated visual data, processing said data using a plurality of machine learning models comprising a natural language processing (NLP) model and an acoustic model to generate an NLP output and an acoustic output, wherein said plurality of machine learning models comprises a neural network trained on labeled conversation data collected from one or more other subjects, wherein said labeled conversation data for each of said one or more other subjects is labeled as (i) having, to some level, said mental or physiological condition or (ii) not having said mental or physiological condition, fusing said NLP output and said acoustic output by (1) applying weights to said NLP output and said acoustic output to generate weighted outputs and (2) generating a composite output from said weighted outputs, wherein said NLP output and said acoustic output each comprise a plurality of outputs corresponding to a plurality of time segments of said conversation data, and wherein said weights in (1) are temporally-based, and outputting an electronic report identifying whether said subject is at risk of having said mental or physiological condition, based at least on said composite output, which risk is quantified in a form of a score having a confidence level provided in said report.

Optionally, the devices, systems, and methods described herein may be used to implement aspect of the devices, systems, and methods in U.S. patent application Ser. No. 17/493,687, titled “Confidence evaluation to measure trust in behavioral health survey results”, filed Oct. 4, 2021, the entirety of which is incorporated by reference herein. Accordingly, the devices, systems, or methods described herein may be interoperable with a method for measuring a degree of confidence in a reliability of responses received from a human subject in a health survey for evaluating a health state of the subject, the method comprising obtaining response data that is generated by the subject in response to prompts presented to the subject during administration of the health survey to the subject, wherein the response data comprises a plurality of conditioning events and a plurality of conditioned events, determining a first probability that a first conditioned event is present in the response data based in part on a presence of a first conditioning event in the response data, wherein the plurality of conditioning events comprises the first conditioning event and the plurality of conditioned events comprises the first conditioned event, and wherein the first probability is used to determine a first event pair comprising the first conditioned event and the first conditioning event, repeating steps the first two steps for two or more other conditioned events and other conditioning events to generate a plurality of additional probabilities for a plurality of additional event pairs, and combining two or more probabilities selected from (i) the first probability and the plurality of additional probabilities or (ii) the plurality of additional probabilities to generate a confidence vector data, wherein the confidence vector data represents a measure of confidence in the reliability of the subject that generated the response data in response to the health survey.

Optionally, the devices, systems, and methods described herein may be used to implement aspect of the devices, systems, and methods in U.S. patent application Ser. No. 17/726,999, titled “Acoustic and natural language processing models for speech-based screening and monitoring of behavioral health conditions”, filed Apr. 22, 2022, the entirety of which is incorporated by reference herein. Accordingly, the devices, systems, or methods described herein may be interoperable with a method for detecting a behavioral or mental health condition in a subject, said method comprising obtaining a speech sample comprising one or more speech segments from said subject, performing at least one of (i) or (ii), wherein (i) comprises processing said speech sample with at least one acoustic model comprising an encoder to generate an acoustic model output comprising an abstract feature representation of said speech sample, wherein said encoder is pretrained to perform a first task other than detecting said behavioral or mental health condition in said subject, and (ii) comprises processing said speech sample, a derivative thereof, and/or said speech sample as transcribed to a text sequence, with at least one natural language processing (NLP) model to generate a language model output, and using at least one of said acoustic model output or said language model output, to individually or jointly generate an output indicative of whether said subject has said behavioral or mental health condition.

Optionally, the devices, systems, and methods described herein may be used to implement aspect of the devices, systems, and methods in PCT Patent Application No. PCT/US2022/015147, titled “Systems and methods for multi-language adaptive mental health risk assessment from spoken and written language”, filed Feb. 3, 2022, the entirety of which is incorporated by reference herein. Accordingly, the devices, systems, or methods described herein may be interoperable with a method for detecting a behavioral or mental health condition, the method comprising receiving an input signal comprising a plurality of audio or lexical characteristics of speech of a subject, wherein at least one of the plurality of audio or lexical characteristics of the speech relates to at least one language, based at least in part on the plurality of audio or lexical characteristics of the input signal, selecting one or more acoustic or natural language processing (NLP) models, wherein at least one of the acoustic or NLP models is a multi-lingual or language-independent model, and detecting a result indicating a presence or absence of the behavioral or mental health condition by processing the input signal with a fused model or joint model derived from the one or more acoustic or NLP models.

### Implementation Details

The foregoing discussion provides many example embodiments. Although each embodiment represents a single combination of inventive elements, other examples may include all possible combinations of the disclosed elements. Thus, if one embodiment comprises elements A, B, and C, and a second embodiment comprises elements B and D, other remaining combinations of A, B, C, or D, may also be used.

The term “connected” or “coupled to” may include both direct coupling (in which two elements that are coupled to each other contact each other) and indirect coupling (in which at least one additional element is located between the two elements).

Throughout the foregoing discussion, numerous references were made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface. The embodiments of the devices, systems and methods described herein can be implemented using, for example, cloud computing, services, and/or edge computing.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

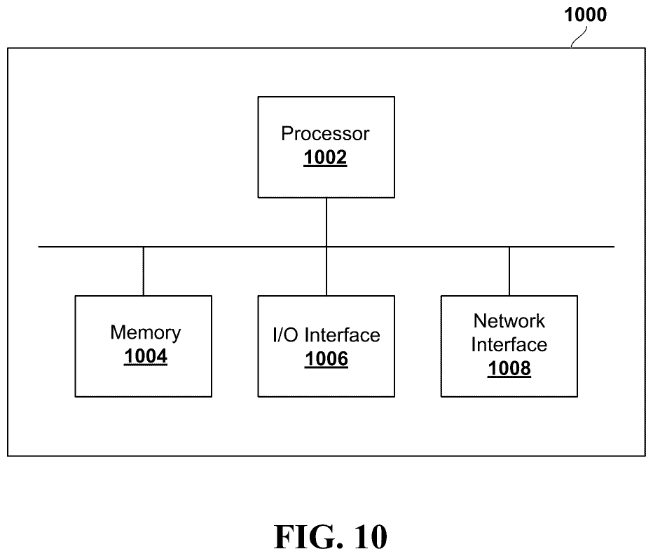

FIG. 10 illustrates a schematic diagram of computing device 1000, according to some embodiments. As depicted, computing device 1000 includes at least one processor 1002, memory 1004, at least one I/O interface 1006, and at least one network interface 1008. Computing device 1000 may be implemented as computing device 104 in system 100.

For simplicity only one computing device 1000 is shown but system may include more computing devices 1000 operable by users to access remote network resources and exchange data. The computing devices 1000 may be the same or different types of devices. The computing device 1000 at least one processor, a data storage device (including volatile memory or non-volatile memory or other data storage elements or a combination thereof), and at least one communication interface. The computing device components may be connected in various ways including directly coupled, indirectly coupled via a network, and distributed over a wide geographic area and connected via a network (which may be referred to as “cloud computing”).

For example, and without limitation, the computing device may be a server, network appliance, set-top box, embedded device, computer expansion module, personal computer, laptop, personal data assistant, cellular telephone, smartphone device, UMPC tablets, video display terminal, gaming console, electronic reading device, and wireless hypermedia device or any other computing device capable of being configured to carry out the methods described herein.

Each processor 1002 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof.

Memory 1004 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 1006 enables computing device 1000 to interconnect with one or more input devices, such as a keyboard, mouse, camera, touch screen and a microphone, or with one or more output devices such as a display screen and a speaker.

Each network interface 1008 enables computing device 1000 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

Computing device 1000 is operable to register and authenticate users (using a login, unique identifier, and password for example) prior to providing access to applications, a local network, network resources, other networks and network security devices. Computing devices 1000 may serve one user or multiple users.

Although the embodiments have been described in detail, it should be understood that various changes, substitutions and alterations can be made herein without departing from the scope as defined by the appended claims.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

As can be understood, the examples described above and illustrated are intended to be exemplary only. The scope is indicated by the appended claims.

What is claimed is:

1. A system for a behavioral or mental health condition of a subject, the system comprising:
at least one input device for receiving conversation data from at least one user;
at least one output device for outputting an electronic report;
at least one computing device in communication with the at least one input device and the at least one output device, the at least one computing device configured to:
receive the conversation data from the at least one input device;
process the conversation data to generate a language model output and/or an acoustic model output;
apply weights to the language model output and/or the acoustic model output, wherein the language model output and the acoustic model output each comprise a plurality of outputs corresponding to a plurality of time segments of the conversation data, and wherein the weights are optionally temporally-based;
generate an electronic report; and
transmit the electronic report to the output device.

2. The system of claim 1, wherein the at least one computing device is further configured to:
fuse the weighted language model output and the acoustic model output generating a composite output.

3. The system of claim 1, wherein the electronic report identifies a severity of at least one symptom of the behavioral or mental health condition based on the composite output.

4. The system of claim 1, wherein the electronic report comprises an annotation of the language model output indicating salience of at least one of the one or more time segments.

5. The system of claim 1, wherein processing the conversation data to generate the language model output and the acoustic model output comprises using a language model neural network and an acoustic neural network trained on labelled conversation data collected from one or more other subjects, wherein the labelled conversation data comprises is labelled as (i) having, to some level, the behavioral or mental health condition and (ii) not having the behavioral or mental health condition.

6. The system of claim 1, wherein the conversation data is processed based in part on at least one of a patient profile and an agent profile, wherein the profile comprises at least one of historical, biographical, demographic, and longitudinal data.

7. The system of claim 1, wherein the conversation data comprises at least one of speech data and text-based data.

8. The system of claim 1, wherein the at least one computing device is configured to run a model based on human-interpretable features.

9. The system of claim 1, wherein the at least one computing device configured to:
determine at least one role of at least one speaker; and
wherein the weights are based in part on the at least one role of the at least one speaker during each time segment.

10. The system of claim 9, wherein the at least one speaker comprises an agent and applying the weights to the language model output and the acoustic model output comprises applying a zero weight to acoustic model output corresponding to the agent.

11. The system of claim 9, wherein the at least one role of the at least one speaker comprises at least one of a patient, an agent, an interactive voice response, and bot or AI speaker.

12. The system of claim 9, wherein the weights applied to the language model output and the acoustic model output are based in part on determining that a number of the at least one speaker matches an expected number of speakers.

13. The system of claim 1, wherein:
the language model output comprises one or more topics corresponding to one or more time ranges, and
the weights are based in part on the one or more topics during each time segment.

14. The system of claim 13, wherein time ranges of the conversation data corresponding to a predefined topic are processed to generate the language model output using more computationally robust models than those used for time ranges of the conversation data not corresponding to the predefined topic.

15. The system of claim 1, wherein the language model output comprises an identification of at least one query based on conversation data from an agent and at least one response to the at least one query based on the conversation data from a patient, wherein the at least one query is mapped onto a predefined query and the at least one response to the at least one query is mapped onto a predefined response to the predefined query.

16. The system of claim 15, wherein the weights are based in part on the at least one query.

17. The system of claim 1, wherein the at least one computing device is further configured to:
receive in-context learning comprising a description of the behavioral or mental health condition;
prior to processing the conversation data to generate the language model output and/or the acoustic model output, pre-processing the conversation data by performing at least one of:
weighing at least one segment of the conversation data based on a relation between the at least one segment of the conversation data and the behavioral or mental health condition;
summarizing the at least one segment of the conversation data;
providing analytics on the at least one segment of the conversation data;
summarizing at least one aspect of the behavioral or mental health condition; and
providing analytics on the at least one aspect of the behavioral or mental health condition; and
transmit the pre-processed conversation data to one or more models to predict the behavioral or mental health condition of the subject.

18. A system for scoring surveys based on a conversation, the system comprising:
at least one input device for receiving conversation data from at least one user;
at least one output device for outputting an electronic report;
at least one computing device in communication with the at least one input device and the at least one output device, the at least one computing device configured to:
receive the conversation data from the at least one input device;
process the conversation data to generate a language model output, wherein the language model output comprises an identification of at least one query based on conversation data from an agent and at least one response to the at least one query based on the conversation data from a patient, wherein the at least one query is mapped onto a predefined query and the at least one response to the at least one query is mapped onto a predefined response to the predefined query; and
generate an electronic report; and
transmit the electronic report to the output device.

19. A system for predicting a severity of at least one symptom of a behavioral or mental health condition of a subject, the system comprising:
at least one input device for receiving conversation data from the subject;
at least one computing device in communication with the at least one input device, the at least one computing device configured to:
receive in-context learning comprising an explanation related to one or more questions of a questionnaire;
receive the conversation data from the at least one input device;
predict the severity of the at least one symptom of the behavioral or mental health condition of the subject based on the in-context learning and the conversation data.
